SEQUENCE LISTING (14 sequences)

SEQ ID NO: 1 - protein, 386 aa, homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 2 - protein, 381 aa, homo sapiens
MRAVPLPLSRTASLSLGFLLLLSLCLDPGQAKELKFVTLVFRHGDRGPIETFPTDPITESSWPQGFGQLTQWGMEQHYELGSYIRKRYGRFLNDTYKHDQIYIRSTDVDRTLMSAMTNLAALFPPEGISIWNPRLLWQPIPVHTVSLSEDRLLYLPFRDCPRFEELKSETLESEEFLKRLHPYKSFLDTLSSLSGFDDQDLFGIWSKVYDPLFCESVHNFTLPSWATEDAMIKLKELSELSLLSLYGIHKQKEKSRLQGGVLVNEILKNMKLATQPQKYKKLVMYSAHDTTVSGLQMALDVYNGVLPPYASCHMMELYHDKGGHFVEMYYRNETQNEPYPLTLPGCTHSCPLEKFAELLDPVISQDWATECMATSSHQGRN

SEQ ID NO: 3 - protein, 381 aa, homo sapiens
MRAVPLHLVGTASLTLGFLLLLSLRLDPGQAKELKFVTLVFRHGDRGPIETFPNDPIKESSWPQGFGQLTKWGMGQHYELGSYIRRRYGRFLNNSYKHDQVYIRSTDVDRTLMSAMTNLAALFPPEGISIWNPRLLWQPIPVHTVSLSEDRLLYLPFRDCPRFQELKSETLKSEEFLKRLQPYKSFIDTLPSLSGFEDQDLFEIWSRLYDPLYCESVHNFTFRTWATEDAMTKLKELSELSLLSLYGIHKQKEKSRLQGGVLVNEILKNMKLATQPQKARKLIMYSAYDTTVSGLQMALELYNGLLPPYASCHIMELYQDNGGTFVEMYYRNETQNEPYPLTLPGCTHSCPLEKFAELLDPVIPQDWATECMGTSNHQASL

SEQ ID NO: 4 - DNA, 24 bp, homo sapiens
tcgtcgttttgtcgttttgtcgtt

SEQ ID NO: 5 - DNA, 4313 bp, homo sapiens
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac

SEQ ID NO: 6 - protein, 920 aa, homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 7 - DNA, 2700 bp, Mus musculus
atggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtga

SEQ ID NO: 8 - protein, 899 aa, Mus musculus
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 9 - DNA, 2709 bp, Rattus
atggaggtgcagttagggctgggaagggtctacccacggcccccgtccaagacctatcgaggagcgttccagaatctgttccagagcgtgcgcgaagcgatccagaacccgggccccaggcaccctgaggccgctagcatagcacctcccggtgcctgtttacagcagcggcaggagactagcccccggcggcggcggcggcagcagcaccctgaggatggctctcctcaagcccacatcagaggcaccacaggctacctggccctggaggaggaacagcagccttcacagcagcagtcagcctccgagggccaccctgagagcggctgcctcccggagcctggagctgccacggctcctggcaaggggctgccgcagcagccaccagctcctccagatcaggatgactcagctgccccatccacgttgtccctactgggccccactttcccaggcttaagcagctgctccgcagacattaaagacatcctgagcgaggccggcaccatgcaacttcttcagcagcagcagcaacagcaacagcagcagcagcagcagcagcagcagcagcagcaacagcagcaggaggtaatatccgaaggcagcagcagcgtgagagcaagggaggccactggggctccctcttcctccaaggatagttacctagggggcaattcgaccatatctgacagtgccaaggagttgtgtaaagcagtgtctgtgtccatggggttgggtgtggaagcactggaacatctgagtccaggggagcagcttcggggcgactgcatgtacgcgtcgctcctgggaggtccacccgccgtgcgtcccactccttgtgcgcctctggccgaatgcaaaggtctttccctggacgaaggcccgggcaaaggcactgaagagactgctgagtattcctctttcaagggaggttacgccaaagggttggaaggtgagagtctgggctgctctggcagcagtgaagcaggtagctctgggacacttgagatcccgtcctcactgtctctgtataagtctggagcagtagacgaggcagcagcataccagaatcgcgactactacaactttccgctcgctctgtccgggccgccgcaccccccgccccctacccatccacacgcccgcatcaagctggagaacccgtcggactacggcagcgcctgggctgcggcggcagcgcaatgccgctatggggacttggctagcctacatggagggagtgtagccggacccagcactggatcgcccccagccaccgcctcttcttcctggcatactctcttcacagctgaagaaggccaattatatgggccaggaggcgggggcggcagcagtagcccaagcgatgctgggcctgtagccccctatggctacactcggccccctcaggggctggcaagccaggagggtgacttctctgcctctgaagtgtggtatcctggtggagttgtgaacagagtcccctatcccagtcccagttgtgttaaaagtgaaatgggaccttggatggagaactactccggaccttatggggacatgcgtttggacagtaccagggaccacgttttacccatcgactattacttcccaccccagaagacctgcctgatctgtggagatgaagcttctggttgtcactacggagctctcacttgtggcagctgcaaggtcttcttcaaaagagctgcggaagggaaacagaagtatctatgtgccagcagaaatgattgcaccattgataaatttcggaggaaaaattgtccatcgtgtcgtctccggaaatgttatgaagcagggatgactctgggagctcgtaagctgaagaaacttggaaatctcaaactacaggaagaaggagaaaactccagtgctggtagccccactgaggacccatcccagaagatgactgtatcacacattgaaggctatgaatgtcaacctatctttcttaatgtcctggaagccattgagccaggagtggtgtgtgccggacatgacaacaaccagcctgattcctttgctgccttgttatctagtctcaacgagcttggcgagagacagcttgtacatgtggtcaagtgggccaaggccttgcctggcttccgcaacttgcatgtggatgaccagatggcagtcattcagtattcctggatgggactgatggtatttgccatgggttggcggtccttcactaatgtcaactctaggatgctctactttgcacctgacctggttttcaatgagtatcgcatgcacaagtctcgaatgtacagccagtgcgtgaggatgaggcacctttctcaagagtttggatggctccagataaccccccaggaattcctgtgcatgaaagcactgctactcttcagcattattccagtggatgggctgaaaaatcaaaaattctttgatgaacttcgaatgaactacatcaaggaacttgatcgcatcattgcatgcaaaagaaaaaatcccacatcctgctcaaggcgcttctaccagctcaccaagctcctggattctgtgcagcctattgcaagagagctgcatcaattcacttttgacctgctaatcaagtcccatatggtgagcgtggactttcctgaaatgatggcagagatcatctctgtgcaagtgcccaagatcctttctgggaaagtcaagcccatctatttccacacacagtga

SEQ ID NO: 10 - protein, 902 aa, Rattus [sequence omitted]

SEQ ID NO: 11 - protein, 9 aa, homo sapiens
LQITPQEFL

SEQ ID NO: 12 - protein, 10 aa, homo sapiens
AMGWRSFTNV

SEQ ID NO: 13 - protein, 9 aa, homo sapiens
SQEFGWLQI

SEQ ID NO: 14 - protein, 8 aa, homo sapiens
CKRKNPTS

(19) United States
(12) Patent Application Publication
(10) Pub. No.: US 2022/0133873 A1
(43) Pub. Date: May 5, 2022

(54) CANCER THERAPY

(71) Applicant: Madison Vaccines Inc., Madison, WI (US)

(72) Inventors: Doug McNeel, Madison, WI (US); Richard R. Lesniewski, Madison, WI (US)

(21) Appl. No.: 17/429,041

(22) PCT Filed: Feb. 7, 2020
(86) PCT No.: PCT/US2020/017172

§ 371 Date: Aug. 6, 2021

Related U.S. Application Data
(60) Provisional application No. 62/802,813, filed on Feb. 8, 2019.

(51) Int. Cl. A61K 39/00; A61K 39/395; A61K 31/4166; A61K 31/4439; A61K 45/06; A61P 35/00

(52) U.S. Cl. CPC A61K 39/001193; A61K 39/3955; A61K 31/4166; A61K 2039/545; A61K 45/06; A61P 35/00; A61K 31/4439

(57) ABSTRACT

Provided herein is technology relating to cancer treatment and prevention and particularly, but not exclusively, to compositions and methods related to therapies for prostate cancer.

### CROSS-REFERENCE TO RELATED APPLICATION

This application claims priority to and the benefit of U.S. Provisional Application No. 62/802,813, filed Feb. 8, 2019, which is hereby incorporated by reference in its entirety.

### FIELD

Provided herein is technology relating to cancer treatment and prevention and particularly, but not exclusively, to compositions and methods related to therapies for prostate cancer.

### BACKGROUND

In the United States (U.S.), prostate cancer is the most common cancer in men. In 2017, the American Cancer Society predicts that there will be around 161,360 new diagnoses of prostate cancer, and that around 26,730 fatalities will occur because of it.

Early stage prostate cancer is typically treated with watchful waiting or monitoring, radical prostatectomy, radiation therapy (alone or in combination with androgen depravation therapy (ADT).

Advanced prostate cancer is treated with ADT. Radical prostatectomy is not currently an option for advanced cases, as it does not treat the cancer that has spread to other parts of the body.

Additional treatments for advanced and aggressive prostate cancer are needed.

### SUMMARY

Provided herein is technology relating to cancer treatment and prevention and particularly, but not exclusively, to compositions and methods related to therapies for prostate cancer.

The compositions and methods described herein provided improved treatment over existing protocols for treatment of prostate cancer. By providing intermittent treatment with androgen receptor antagonists in combination with one or more DNA vaccines and PD-1 blockade, the methods described herein allow subjects with advanced prostate cancer to avoid or delay the side effects of testosterone reducing therapies and thus provide improved quality of life.

For example, in some embodiments, provided herein is a method for treating prostate cancer in a subject (e.g, human subject), the method comprising: (a) administering to a subject at least one vaccine comprising a nucleic acid comprising a nucleotide sequence selected from the group consisting of a prostatic acid phosphatase (PAP) gene and a ligand-binding domain of an androgen receptor (AR) gene; (b) administering to the subject a human programmed death receptor-1 (PD-1) inhibitor; and (c) administering to the subject an androgen receptor antagonist, wherein the vaccine, the PD-1 inhibitor, and the androgen receptor antagonist are administered concurrently.

Further embodiments provide the use of a vaccine comprising a nucleic acid comprising a nucleotide sequence from a prostatic acid phosphatase (PAP) gene and/or a AR gene, a human programmed death receptor-1 (PD-1) inhibitor, and an androgen receptor antagonist to treat prostate cancer in a subject in need thereof, wherein the vaccine, the PD-1 inhibitor, and the androgen receptor antagonist are administered concurrently.

In some embodiments, the concurrent administration comprises administration of the vaccine followed by the administration of the PD-1 inhibitor and the androgen receptor antagonist within 24 hours of administration of the vaccine.

The present invention is not limited to particular PAP or AR genes. In some embodiments, the PAP or AR gene is a human or rodent PAP gene. In some embodiments, the nucleotide sequence encodes a polypeptide comprising an amino acid sequence from SEQ ID NO: 1, SEQ ID NO: 2, SEQ ID NO: 3, SEQ ID NO:6, or a portion or substituted variant thereof. In some embodiments, the nucleic acid comprises pTVG4-HP. In some embodiments, the nucleic acid further comprises a transcriptional regulatory element and/or an immunostimulatory sequence. In some embodiments, the nucleotide sequence from a PAP or AR gene is operatively linked to a transcriptional regulatory element.

The present invention is not limited to particular PD-1 inhibitors. In some embodiments, the PD-1 inhibitor is a monoclonal antibody (e.g., pembrolizumab, JNJ-63723283, or nivolumab). In some embodiments, the PD-1 inhibitor is administered at a dose of 1 to 5 mg/kg. In some embodiments, the PD-1 inhibitor is administered at a dose of from 150 to 250 mg, preferably from 180 to 200 mg and most preferably about 200 mg every three weeks or every 4 weeks or from 220 to 260 mg and most preferably about 240 mg every 2 weeks or from 450 to 510 mg and most preferably 480 mg every 4 weeks. In some embodiments, the PD-1 inhibitor is administered intravenously.

The present invention is not limited to particular androgen receptor antagonists. In some embodiments, the androgen receptor antagonist is enzalutamide or apalutamide. In some embodiments, enzalutamide is administered at a dose of from 120 to 200 mg, preferably from 140 to 180 mg, and most preferably about 160 mg daily or from 210 to 270 mg and more preferably about 240 mg daily and said apalutamide is administered at a dose of from 210 to 270 mg and more preferably about 240 mg daily.

In some embodiments, the vaccine further comprises an adjuvant (e.g., GM-CSF). In some embodiments, the vaccine is administered intradermally or transdermally. In some embodiments, the vaccine is administered in an amount of approximately 100 μg. In some embodiments, the vaccine is administered about every 1 to 4 (e.g., every 3 weeks). In some embodiments, the vaccine, the androgen receptor antagonist, and the PD-1 inhibitor are administered a plurality of times, and wherein after the first concurrent administration of the vaccine and the PD-1 inhibitor, the vaccine is administered every 10 to 21 days, the PD-1 inhibitor is administered every 17 to 30 days for a period of up to 90 days, and the androgen receptor antagonist is administered daily for a period of up to 90 days beginning 1 to 16 weeks, and most preferably about 12 weeks after the first administration of the vaccine. In some embodiments, androgen receptor antagonist is administered daily for 90 days followed by a period of not administering the androgen receptor antagonist. For example, in some embodiments, the daily administration of the androgen receptor antagonist is repeated every 90 days after a 90 day rest period where the androgen receptor antagonist is not administered. In some embodiments, the vaccine and the PD-1 inhibitor are administered concurrently every 10 to 28 days for a period of up to 90 days. In some embodiments, the vaccine and the PD-1 inhibitor are administered every 10 to 28 days for a period of from 91 days to 365 days or a period of from 366 days to 730 days. In some embodiments, the vaccine comprises a first vaccine to PAP and a second vaccine to AR and the first and second vaccine are administered concurrently (e.g., in separate or the same pharmaceutical compositions).

In some embodiments, the method or use produces an anti-tumor response in the subject that is improved relative to administration of the vaccine alone or the vaccine in combination with the PD-1 inhibitor. In some embodiments, the method or use increases the number of PAP and/or AR-specific T cells or antibodies in the subject. In some embodiments, the method or use results in undetectable PSA levels (e.g., that persist after discontinuation of administration of the androgen receptor antagonist (e.g., for at least 1, 4, 6, 12, 24 or more months after discontinuation of administration of the androgen receptor antagonist).

Further embodiments provide a kit comprising: 1) a first pharmaceutical composition comprising a vaccine comprising a nucleic acid comprising a nucleotide sequence selected from the group consisting of a prostatic acid phosphatase (PAP) gene and a ligand-binding domain of an androgen receptor (AR) gene; 2) a second pharmaceutical composition comprising a PD-1 inhibitor; 3) a third pharmaceutical composition comprising an androgen receptor antagonist. In some embodiments, the first, second, and third pharmaceutical compositions are provided as single doses. In some embodiments, the kit comprises an amount of the first pharmaceutical composition, an amount of the second pharmaceutical composition, and an amount of the third pharmaceutical composition sufficient to provide enough doses for a dosing schedule in which the nucleic acid vaccine, the PD-1 inhibitor, and androgen receptor antagonist are administered multiple times.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

FIG. 1A shows best % change in serum PSA from day 1 of study. Asterisks indicate those patients who had evidence of PAP-specific Th1 immunity (significant IFN and/or granzyme B response detected at least twice post-treatment).

FIG. 1B shows best change from baseline for serum PSA over 6 months (left) and 24 months (right).

FIG. 2 shows an exemplary clinical study protocol.

FIG. 3 shows exemplary PAP sequences.

FIG. 4 shows exemplary AR sequences.

FIG. 5A-B show progression free survival in subjects with and without immune response to A) AR peptide; and B) AR protein.

FIG. 6A-B shows patient response to AR in subjects with and without response to AR for AR vaccine in combination with ADT. C) PCWG2 criteria; D) time to PSA progression in subjects with and without response to AR.

FIG. 7A-B shows that AR vaccine plus checkpoint inhibitors provide improved anti-tumor effects in mice bearing prostate tumors. A) AR vaccine plus PD-1 inhibitor. B) AR vaccine and ADT plus PD-1 inhibitor.

### DETAILED DESCRIPTION

Provided herein is technology relating to cancer treatment and prevention and particularly, but not exclusively, to compositions and methods related to therapies for prostate cancer. In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the “best overall response” is the best response recorded from baseline until disease progression/recurrence, taking as reference for progressive disease the smallest measurements recorded after baseline.

As used herein, the “first documentation of response” refers to the time between initiation of therapy and first documentation of a partial response or complete response to therapy as defined herein.

As used herein, the “duration of response” refers to the period measured from the time that measurement criteria are met for complete or partial response (whichever status is recorded first) until the first date that recurrent or progressive disease is objectively documented, taking as reference the smallest measurements recorded since treatment started.

As used herein, the “duration of overall complete response” refers to the period measured from the time measurement criteria are met for complete response until the first date that recurrent disease is objectively documented.

As used herein, the “duration of stable disease” refers to a measurement from baseline until the criteria for disease progression is met, taking as reference the smallest measurements recorded since baseline.

As used herein, “survival” refers to the time interval from initiation of a treatment according to the technology described to death from any cause or to the last follow-up in censored patients.

As used herein, the terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

The term “portion” when used in reference to a protein (as in “a portion of a given protein”) refers to fragments of that protein, such as “peptides” of the protein. The fragments may range in size from four amino acid residues to the entire amino sequence minus one amino acid (for example, the range in size includes 4, 5, 6, 7, 8, 9, 10, or more amino acids up to the entire amino acid sequence minus one amino acid).

The term “homolog” or “homologous” when used in reference to a polypeptide refers to a high degree of sequence identity between two polypeptides, or to a high degree of similarity between the three-dimensional structure, or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (e.g., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The nomenclature used to describe variants of nucleic acids or proteins specifies the type of mutation and base or amino acid changes. For a nucleotide substitution (e.g., 76A>T), the number is the position of the nucleotide from the 5′ end, the first letter represents the wild type nucleotide, and the second letter represents the nucleotide which replaced the wild type. In the given example, the adenine at the 76th position was replaced by a thymine. If it becomes necessary to differentiate between mutations in genomic DNA, mitochondrial DNA, complementary DNA (cDNA), and RNA, a simple convention is used. For example, if the 100th base of a nucleotide sequence is mutated from G to C, then it would be written as g·100G>C if the mutation occurred in genomic DNA, m·100G>C if the mutation occurred in mitochondrial DNA, c·100G>C if the mutation occurred in cDNA, or r·100 g>c if the mutation occurred in RNA. For amino acid substitution (e.g., D111E), the first letter is the one letter code of the wild type amino acid, the number is the position of the amino acid from the N-terminus, and the second letter is the one letter code of the amino acid present in the mutation. Nonsense mutations are represented with an X for the second amino acid (e.g. D111X). For amino acid deletions (e.g. ΔF508, F508del), the Greek letter A (delta) or the letters “del” indicate a deletion. The letter refers to the amino acid present in the wild type and the number is the position from the N terminus of the amino acid where it is present in the wild type. Intronic mutations are designated by the intron number or cDNA position and provide either a positive number starting from the G of the GT splice donor site or a negative number starting from the G of the AG splice acceptor site. g·3′+7G>C denotes the G to C substitution at nt+7 at the genomic DNA level. When the full-length genomic sequence is known, the mutation is best designated by the nucleotide number of the genomic reference sequence. See den Dunnen & Antonarakis, “Mutation nomenclature extensions and suggestions to describe complex mutations: a discussion”. Human Mutation 15: 7-12 (2000); Ogino S, et al., “Standard Mutation Nomenclature in Molecular Diagnostics: Practical and Educational Challenges”, J. Mol. Diagn. 9(1): 1-6 (February 2007), incorporated herein by reference in their entireties for all purposes.

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include the transmembrane domains, and the glycosylation sites.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression can be regulated at many stages in the process.

As used herein, the term “operably linked” is intended to mean that the transcription or translation of a nucleotide sequence is under the influence of another functional nucleotide sequence, such as a promoter, an enhancer, a transcription factor binding site, etc. “Operably linked” is also intended to mean the joining of two nucleotide sequences such that the coding sequence of each DNA fragment remains in the proper reading frame. In this manner, the nucleotide sequences for promoters, enhancers, etc. are provided in DNA constructs along with the nucleotide sequence of interest, e.g., a nucleotide sequence encoding PAP, for expression in the subject. The term “heterologous nucleotide sequence” is intended to mean a sequence that is not naturally operably linked with the promoter sequence. While this nucleotide sequence is heterologous to the promoter sequence, it may be homologous (“native”) or heterologous (“foreign”) to the subject.

As used herein, the term “treatment” is defined as the application or administration of a therapeutic agent described herein (e.g., a DNA vaccine, a PD-1 inhibitor, and/or an androgen receptor antagonist), or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Compositions according to the technology can be administered in the form of pharmaceutically acceptable salts. The term “pharmaceutically acceptable salt” refers to a salt that possesses the effectiveness of the parent compound and is not biologically or otherwise undesirable (e.g., is neither toxic nor otherwise deleterious to the recipient thereof). Suitable salts include acid addition salts that may, for example, be formed by mixing a solution of the compound of the present technology with a solution of a pharmaceutically acceptable acid such as hydrochloric acid, sulfuric acid, acetic acid, trifluoroacetic acid, or benzoic acid. Certain of the compounds employed in the present technology may carry an acidic moiety (e.g., COOH or a phenolic group), in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (COOH) or alcohol group being present, pharmaceutically acceptable esters can be employed to modify the solubility or hydrolysis characteristics of the compound. For example, pharmaceutically acceptable salts include both the metallic (inorganic) salts and organic salts, a list of which is given in Remington's Pharmaceutical Sciences, 17th Edition, pg. 1418 (1985). It is well known to one skilled in the art that an appropriate salt form is chosen based on physical and chemical properties. As will be understood by those skilled in the art, pharmaceutically acceptable salts include, but are not limited to salts of inorganic acids such as hydrochloride, sulfate, phosphate, diphosphate, hydrobromide, and nitrate; or salts of an organic acid such as malate, maleate, fumarate, tartrate, succinate, citrate, acetate, lactate, methanesulfonate, p-toluenesulfonate or palmoate, salicylate, and stearate. Similarly pharmaceutically acceptable cations include, but are not limited to sodium, potassium, calcium, aluminum, lithium, and ammonium (especially ammonium salts with secondary amines). Also included within the scope of this technology are crystal forms, hydrates, and solvates.

The term “administration” and variants thereof (e.g., “administering” a compound, vaccine, drug, etc.) in reference to a compound mean providing the compound or a prodrug of the compound to the individual in need of treatment or prophylaxis. When a compound of the technology or a prodrug thereof is provided in combination with one or more other active agents, “administration” and its variants are each understood to include provision of the compound or prodrug and other agents at the same time or at different times. As used herein, the term “concurrent administration” refers to the administration of two agents, preferably within 24 hours of one another. When the agents of a combination are administered “concurrently” (e.g., within 24 hours of one another), they can be administered together in a single composition or they can be administered separately. In instances where they are administered separately, the first agent such as a PAP vaccine, is administered, the patient is monitored, and then the second agent such as PD-1 inhibitor and androgen receptor antagonist is administered within a specified time period, preferably 24 hours. As used herein, the term “composition” is intended to encompass a product comprising the specified ingredients in the specified amounts, as well as any product that results, directly or indirectly, from combining the specified ingredients in the specified amounts.

As used herein, the terms “co-administration” and “co-administering” refer to the administration of at least two agent(s) (e.g., a DNA vaccine and a PD-1 inhibitor and/or an androgen receptor antagonist) or therapies to a subject. In some embodiments, the co-administration of two or more agents or therapies is concurrent. In other embodiments, a first agent/therapy is administered prior to a second agent/therapy. In some embodiments, co-administration can be via the same or different route of administration. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutically acceptable” means that the ingredients of the pharmaceutical composition are compatible with each other and not deleterious to the recipient thereof.

The term “subject” as used herein refers to an animal, preferably a mammal, most preferably a human, who has been the object of treatment, observation, or experiment.

The term “effective amount” as used herein means that amount of active compound or pharmaceutical agent that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptoms of the disease or condition being prevented. When the active compound is administered as the salt, references to the amount of active ingredient are to the free form (the non-salt form) of the compound.

In the method of the present technology, compounds, optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the technology can, for example, be administered orally, parenterally (including subcutaneous injections, intravenous, intramuscular, intrasternal injection, or infusion techniques), by inhalation spray, or rectally, in the form of a unit dosage of a pharmaceutical composition containing an effective amount of the compound and conventional non-toxic pharmaceutically-acceptable carriers, adjuvants, and vehicles. Liquid preparations suitable for oral administration (e.g., suspensions, syrups, elixirs, and the like) can be prepared according to techniques known in the art and can employ any of the usual media such as water, glycols, oils, alcohols, and the like. Solid preparations suitable for oral administration (e.g., powders, pills, capsules, and tablets) can be prepared according to techniques known in the art and can employ such solid excipients as starches, sugars, kaolin, lubricants, binders, disintegrating agents, and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution, or a solution containing a mixture of saline and glucose. Further description of methods suitable for use in preparing pharmaceutical compositions for use in the present technology and of ingredients suitable for use in the compositions is provided in Remington's Pharmaceutical Sciences, 18th edition, edited by A. R. Gennaro, Mack Publishing Co., 1990. Compounds of the present technology can be made by a variety of methods depicted in the synthetic reaction schemes provided herein. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis, Wiley & Sons: New York, Volumes 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40.

As used herein, the term “a composition for inducing an immune response” refers to a composition that, once administered to a subject (e.g., once, twice, three times or more (e.g., separated by weeks, months or years)), stimulates, generates, and/or elicits an immune response in the subject (e.g., resulting in the production of CD8+ and or CD4+ T-cells and/or the production of antibodies). In some embodiments, the composition comprises a nucleic acid and one or more other compounds or agents including, but not limited to, therapeutic agents, physiologically tolerable liquids, gels, carriers, diluents, adjuvants, excipients, salicylates, steroids, immunosuppressants, immunostimulants, antibodies, cytokines, antibiotics, binders, fillers, preservatives, stabilizing agents, emulsifiers, and/or buffers. An immune response may be an innate (e.g., a non-specific) immune response or a learned (e.g., acquired) immune response.

As used herein, the term “adjuvant” refers to any substance that can stimulate an immune response. Some adjuvants can cause activation of a cell of the immune system (e.g., an adjuvant can cause an immune cell to produce and secrete a cytokine). Examples of adjuvants that can cause activation of a cell of the immune system include, but are not limited to, Granulocyte-macrophage colony-stimulating factor (GM-CSF), saponins purified from the bark of the Q. saponaria tree, such as QS21 (a glycolipid that elutes in the 21st peak with HPLC fractionation; Aquila Biopharmaceuticals, Inc., Worcester, Mass.); poly(di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). Traditional adjuvants are well known in the art and include, for example, aluminum phosphate or hydroxide salts (“alum”). In some embodiments, compositions of the present technology are administered with one or more adjuvants.

As used herein, the term “an amount effective to induce an immune response” (e.g., of a composition for inducing an immune response), refers to the dosage level required (e.g., when administered to a subject) to stimulate, generate, and/or elicit an immune response in the subject. An effective amount can be administered in one or more administrations (e.g., via the same or different route), applications, or dosages and is not intended to be limited to a particular formulation or administration route.

As used herein, the term “under conditions such that said subject generates an immune response” refers to any qualitative or quantitative induction, generation, and/or stimulation of an immune response (e.g., innate or acquired).

A used herein, the term “immune response” refers to a response by the immune system of a subject. For example, immune responses include, but are not limited to, a detectable alteration (e.g., increase) in Toll-like receptor (TLR) activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen presenting cells. An immune response may be to immunogens that the subject's immune system recognizes as foreign (e.g., non-self antigens or self-antigens recognized as foreign). Thus, it is to be understood that, as used herein, “immune response” refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen as well as acquired (e.g., memory) responses that are a result of an adaptive immune response).

As used herein, the terms “immunogen” and “antigen” refer to an agent (e.g., a PAP and/or AR polypeptide) and/or portions or components thereof (e.g., a peptide from a PAP polypeptide and or the AR polypeptide) that is capable of eliciting an immune response in a subject.

As use herein, the term “disease progression” refers to the appearance of new evidence of advancement of disease by a diagnostic assay such as a molecular assay or imaging assay, for example, the appearance of new lesions on bone scan.

### DESCRIPTION

Although the disclosure herein refers to certain illustrated embodiments, it is to be understood that these embodiments are presented by way of example and not by way of limitation.

Dramatic clinical responses observed with PD-1/PD-L1 inhibitors was in part one of the reasons cancer immunotherapy was named the scientific breakthrough of the year in 2013 by Science [1]. In fact, targeting PD-1 may be a universal anti-cancer therapy, as it targets T-cells rather than tumors directly. However, clinical trial experience to date indicates that patients with some solid tumor types experience more benefit than patients with other histologies, including prostate cancer [2]. This disparity is likely due to differences in the preexisting T-cells of responding and non-responding patients. It has been demonstrated that prostate cancers can express PD-L1 and have infiltrating PD-1-expressing T-cells, although these are at low frequencies relative to some cancers [3]. Taken together, these results indicate that the efficacy of PD-1/PD-ligand blockade may be increased for less responsive tumors (including prostate cancer) by combining with treatments aimed at increasing the frequency of functionally active, tumor-specific CD8+ T-cells.

Patients with early, PSA-recurrent (non-metastatic) prostate cancer were evaluated for response to a DNA vaccine targeting prostatic acid phosphatase (MV-816) [4, 5]. In 38 patients treated, no significant adverse events were observed. Moreover, several patients developed evidence of PAP-specific CD4+ and CD8+ T-cells, and several patients experienced a prolongation in the PSA doubling time, demonstrating immunological efficacy and indicating a possible anti-tumor effect [4, 5]. The presence of long-term IFNγ-secreting immune responses to PAP, detectable at multiple times months after immunization, were associated with increases in PSA doubling time, indicating this may serve as a rational biomarker for efficacy [6]. Moreover, it was found that immune responses could be augmented months later with repeated immunizations, indicating that DNA vaccines might provide a simple means of eliciting tumor-specific CD8+ T-cells [5]. Further preliminary studies have shown that patients previously treated with MVI-816 have EpCam+ circulating epithelial cells (CEC) with PD-L1 expression, analogous to findings in murine models [7]. These findings further support that the MVI-816 vaccine can be used to elicit CD8+ T-cells specific for prostate tumors, the efficacy of which may be augmented with concurrent treatment with PD-1 blockade.

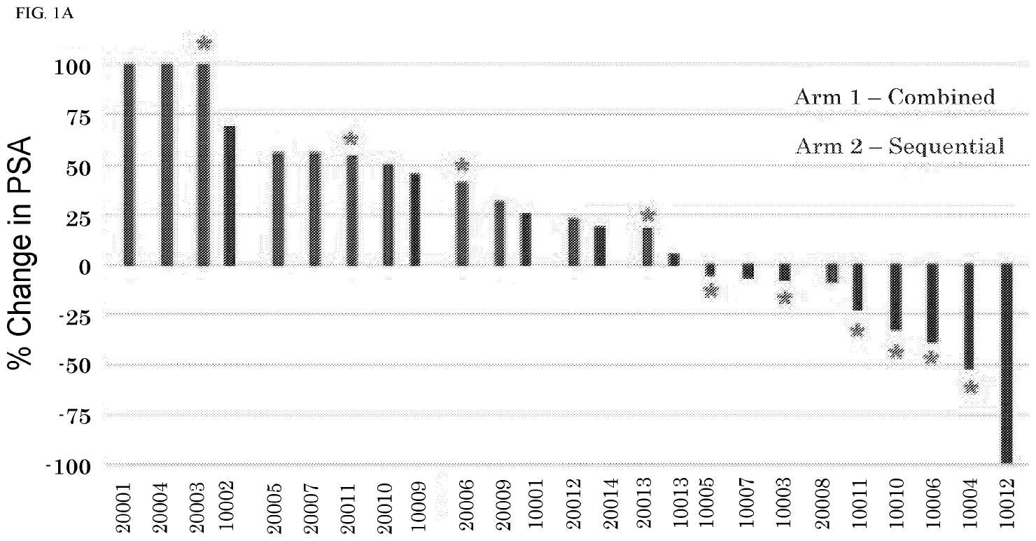

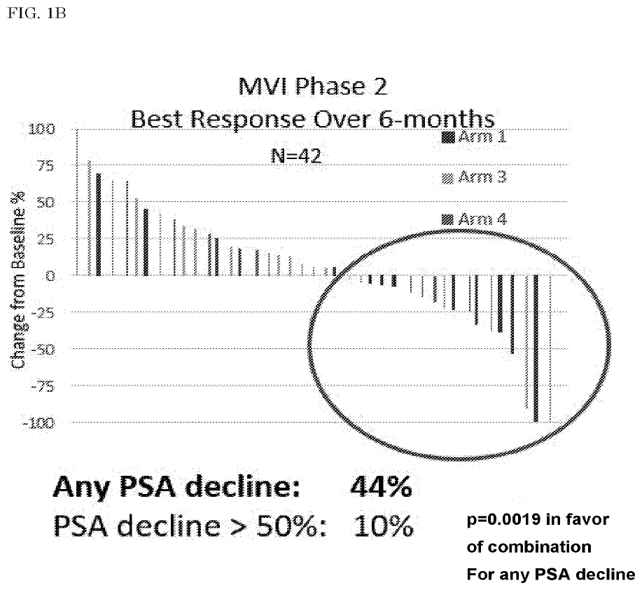

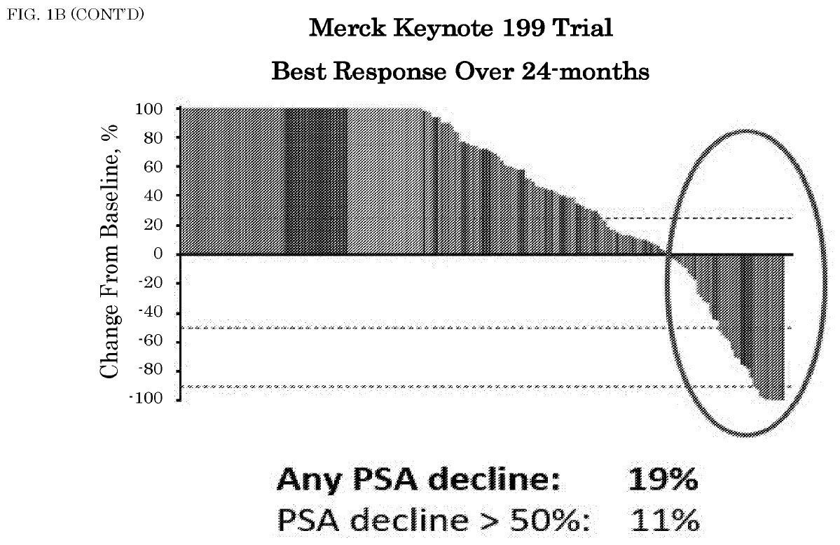

Studies conducted during the course of development of embodiments of the present disclosure demonstrated that T-cell activation by DNA vaccination leads to PD-1 upregulation on CD8+ T-cells, and that blockade of PD-1 at the time of vaccination leads to better anti-tumor responses in murine models. Based on these findings, a study was conducted using MVI-816 in combination with pembrolizumab in patients with metastatic, castration-resistant prostate cancer (mCRPC). In this trial, patients received both agents, together or in sequence, over a 12-to-24-week course. It was observed that PD-1 blockade with pembrolizumab, when administered concurrently with vaccine, elicited PSA declines in 8 of the 13 patients (FIG. 1), and objective tumor responses were seen in two of the patients with greatest PSA declines. PSA responses were associated with the development of immune response to the PAP target antigen, and elicited CD8+ tumor-infiltrating lymphocytes, consistent with the role of vaccines as T-cell activating therapies. These are encouraging findings, notably because PD-1 blockade with checkpoint inhibitors alone has demonstrated little single-agent clinical activity in patients with this stage of prostate cancer in previous phase I trials, with the possible exception of patients concurrently receiving enzalutamide [8, 9, 10]. Moreover, PSA declines and objective radiographic responses are rare following vaccine therapies.

Androgen deprivation is the cornerstone of treatment for patients with metastatic prostate cancer. It is also commonly used in patients with PSA-recurrent prostate cancer, and in this stage of disease has been used on either an intermittent or continuous basis. In addition to its on-target direct antitumor effects, androgen deprivation also has immunostimulatory effects. These include the induction of thymic regrowth and increased release of naïve T-cells, an increase in immune cell infiltration into the prostate (both myeloid and lymphocyte populations), decreased numbers of regulatory T-cells, and increased antibody responses to prostate antigens (11-16). Preclinical studies have shown that androgen deprivation can enhance the efficacy of various immunotherapeutic approaches, including checkpoint blockade (17), irradiated tumor cell vaccines (18), T-cell adoptive transfer (19), and antigen-specific vaccines (20, 21).

Recently published results (22) showed that androgen deprivation increased AR expression in human and murine prostate tumor cells in vitro and in vivo. The increased expression persisted over time. Increased AR expression was associated with recognition and cytolytic activity by AR-specific T-cells. Furthermore, ADT combined with vaccination, specifically a DNA vaccine encoding the ligand-binding domain of the AR (MVI-118), led to improved antitumor responses as measured by tumor volumes and delays in the emergence of castrate-resistant prostate tumors in two murine prostate cancer models (Myc-CaP and prostate-specific PTEN-deficient mice).

Accordingly, in some embodiments, ADT is combined with AR-directed immunotherapy to target a major mechanism of resistance, overexpression of the AR. In some embodiments, enzalutamide, an androgen receptor antagonist, is used in combination with anti-tumor vaccination [21]. An advantage of androgen receptor antagonists, including enzalutamide or apalutamide, is that they mediate their effects without affecting testosterone production. Thus, they can be used intermittently, combined with immune therapy as described [23], sparing the potential long-term effects of testosterone suppression in patients with earlier stages of disease. The ability to drive PSA to undetectable levels, possibly curing or significantly delaying the metastatic recurrence of prostate cancer prior to the need for androgen depriving therapies, is a substantial and clinically meaningful “game-changing” advance in the therapy of this disease.

Exemplary compositions and methods are described herein.

### I. DNA Vaccines

Embodiments of the present invention comprising DNA vaccines against cancer targets (e.g., PAP and/or AR).

### PAP

Prostatic acid phosphatase (PAP) is a tumor antigen in prostate cancer and PAP-specific CD8+ CTL can lyse prostate cancer cells. PAP was first identified in 1938 and was initially used as a serum marker for the detection of prostate cancer. PAP expression in normal and malignant prostate cells is well-documented, and is still used in immunohistochemical staining to establish a prostate origin of metastatic carcinoma. The ubiquitous expression of PAP in prostate tissue makes it an appealing antigen as a potential “universal” target for immune-directed therapies of prostate cancer, unlike specific oncogenes that may or may not be expressed by a particular tumor. Moreover, it has been demonstrated that some patients with prostate cancer have preexisting antibody and T-cell responses to PAP, suggesting that tolerance to this “self” protein can be circumvented in vivo. In particular, Th1-like immune responses specific for PAP indicate that an immune environment permissive of an anti-tumor response exists in patients even without immunization. Moreover, experiments have previously demonstrated that CD8+ T cells specific for PAP, with cytolytic activity for prostate cancer cells, exist in patients with prostate cancer and can be augmented with vaccination.

PAP genes are known and have been cloned from human, mouse, and rat (see, e.g., SEQ ID NO: 1, SEQ ID NO: 2, and SEQ ID NO: 3, respectively). As will be readily recognized by one of ordinary skill in the art, any DNA sequence that encodes a PAP gene is suitable for the present invention, and any other PAP genes from other animals, as they become identified, characterized, and cloned are also suitable for the present invention. Dogs and non-human primates are known to have PAP genes. It is readily recognizable that a PAP gene of any origin, or any of its derivatives, equivalents, variants, mutants etc., is suitable for the instant technology, as long as the protein encoded by the genes, or derivatives, equivalents, variants, or mutants thereof induce an immune reaction in the host animal substantially similar to that induced by an autoantigenic or xenoantigenic PAP protein in the animal. The role of PAP in prostate cancer and exemplary vaccines that target PAP are described in WO 2017/139628, herein incorporated by reference in its entirety.

### AR

In some embodiments, provided herein are DNA vaccines that target the ligand binding domain of an androgen receptor are provided (See e.g., U.S. Pat. Nos. 9,433,668; 8,962,590; 8,513,210; and 7,910,565; each of which are herein incorporated by reference in their entities). Such vaccines utilize the ligand-binding domain of an androgen receptor of any origin, or any of the ligand-binding domain's derivatives, equivalents, variants, mutants etc., is suitable for the instant invention, as long as the ligand-binding domain or derivatives, equivalents, variants, or mutants thereof is able to induce an immune reaction in the host human or non-human animal substantially similar to that induced by an autoantigenic or xenoantigenic ligand-binding domain of the androgen receptor in the animal. Similarly, a polynucleotide sequence of an androgen receptor gene of any origin that encodes the ligand-binding domain of the receptor, or any of the polynucleotide's derivatives, equivalents, variants, mutants etc., is suitable for the instant invention, as long as the polynucleotide sequence and the polypeptide or protein encoded by the polynucleotide sequence, or derivatives, equivalents, variants, or mutants thereof is able to induce an immune reaction in the host human or non-human animal substantially similar to that induced by an autoantigenic or xenoantigenic ligand-binding domain of the androgen receptor in the animal.

Androgen receptor genes are known and have been cloned from many species. For example, the human, mouse, rat, dog, chimpanzee, macaque, and lemur androgen receptor cDNA along with amino acid sequences can be found at GenBank Accession Nos. NM_000044 (cDNA-SEQ ID NO:5 and amino acid sequence-SEQ ID NO:6), NM_013476 (cDNA-SEQ ID NO:7 and amino acid sequence-SEQ ID NO:8), NM_012502 (cDNA-SEQ ID NO:9 and amino acid sequence-SEQ ID NO:10), NM_001003053, NM_001009012, U94179, and U94178, respectively. Androgen receptor genes from other species are also known. These species include but are not limited to Sus scrofa, Astatotilapia burtoni, Gallus gallus, Kryptolebias marmoratus, Alligator mississippiensis, Leucoraja erinacea, Haplochromis burtoni, Pimephales promelas, Dicentrarchus labrax, Gambusia affinis, Micropogonias undulates, Oryzias latipes, Acanthopagrus schlegelii, Rana catesbeiana, Crocuta crocuta, Eulemur fulvus collaris, and Anguilla japonica (see GenBank Accession Nos. NM_214314 (or AF161717), AY082342, NM_001040090, DQ339105, AB186356, DQ382340, AF121257, AY727529, AY647256, AB099303, AY701761, AB076399, AY219702, AY324231, AY128705, U94178, and AB023960, respectively). For the purpose of the present invention, the ligand-binding domain of the human androgen receptor refers to a polypeptide that starts at any amino acid from amino acid positions 651 to 681 and ends at any amino acid from amino acid positions 900 to 920. For example, human androgen receptor or a fragment of the human androgen receptor that comprises amino acids 681-900 as well as DNA vaccines containing a polynucleotide encoding the above are suitable vaccines. The corresponding ligand-binding domains of androgen receptors from other species can be readily determined by sequence alignment (to the human sequence) (e.g., by the methods described below in connection with sequence identity or homology). In a preferred embodiment, a polypeptide from the human androgen receptor that starts at any amino acid from amino acid positions 661 to 671 and ends at any amino acid from amino acid positions 910 to 920 is used in the present invention. In a more preferred embodiment, a polypeptide containing amino acids 661 to 920 or 664 to 920 of the human androgen receptor is used in the present invention. To help determine the corresponding fragments of the androgen receptors from other species, it is noted here that the amino acid positions on rat, dog, chimpanzee, macaque, and lemur androgen receptors that correspond to amino acid positions 661 to 920 of the human androgen receptor are 640 to 899, 643 to 902, 648 to 907, 652 to 910, 636 to 895, and 625 to 884, respectively. It is noted that the above fragments of the human, mouse, rat, dog, chimpanzee, macaque, and lemur androgen receptors have the same amino acid sequence. The ligand-binding domains of the androgen receptors of other species are also known or can be readily identified through sequence alignment. As will be readily recognized by one of ordinary skill in the art, any DNA sequence that encodes a ligand-binding domain or a larger fragment of an androgen receptor including the full-length receptor from one of the above species as well as other animals is suitable for the present invention.

In addition, fragments of a ligand binding domain of an androgen receptor such as those that can bind to HLA-A2 are also useful antigens which elicit cytotoxic responses against cells expressing the androgen receptor or its ligand binding domain. Polynucleotides that encode these fragments are considered functional equivalents. Examples of these fragments are provided in the examples below. In particular, the use of the following four fragments are contemplated: SEQ ID NO:11 (amino acids 811-819 of SEQ ID NO:6), SEQ ID NO:12 (amino acids 761-770 of SEQ ID NO:6), SEQ ID NO:13 (amino acids 805-813 of SEQ ID NO:6), and SEQ ID NO:14 (amino acids 859-867 of SEQ ID NO:6).

### DNA Vaccines

The present invention provides DNA-based vaccines that express a polypeptide antigen, the ligand-binding domain of a mammalian androgen receptor or certain fragments thereof, and/or a PAP antigen, and methods for treating prostate cancers in a human or non-human animal using the vaccines. In some embodiments, vaccines are plasmid vaccines. An advantage of plasmid DNA vaccines is that they encode a defined, often small, number of proteins. Therefore, one can repetitively immunize the animal or patient. Furthermore, a virus may kill cells, incorporate into the genome, or potentially induce other unwanted immune responses. All these are disadvantages that are likely avoided by DNA plasmid vaccines. In some embodiments, the DNA vaccine technology (e.g., relating to compositions, methods, etc.) is as described in U.S. Pat. App. Pub. No. 20040142890 A1, which is explicitly incorporated herein by reference in its entirety.

DNA vaccines, like peptide-based vaccines, are advantageous in being relatively easy and inexpensive to manufacture, and are not individualized for patients, as are dendritic cell-based vaccines. Unlike recombinant protein vaccines, in which the antigen is taken up by antigen presenting cells and expressed predominantly in the context of MHC class II, DNA in nucleic acid vaccines is taken up and expressed by antigen-presenting cells directly, leading to antigen presentation through both naturally processed MHC class I and II epitopes [38].

In some embodiments, the derivatives, equivalents, variants, fragments, or mutants of a PAP polypeptide are at least 85% identical in sequence to the human PAP sequence of SEQ ID NO:1 or the AR sequence of SEQ ID NO:6. More preferably, the identity is at least 88%, preferably at least 90%, still more preferably at least 95%, and still more preferably at least 95%. Identity between amino acid sequences or between nucleotide sequences may be determined either manually by one skilled in the art, or by using computer-based sequence comparison and identification tools that employ algorithms such as BLAST (Basic Local Alignment Search Tool; Altschul et al. (1993) J. Mol. Biol. 215:403-410).

In some embodiments, fragments of the full-length genes which encode portions of the full-length PAP or AR protein are constructed. These fragments peptides that elicits humoral or cytotoxic reaction, or both, against the protein antigen, and are considered functional equivalents.

The present technology provides DNA-based vaccines that express a protein antigen, prostatic acid phosphatase (PAP) and/or an AR antigen. In some embodiments, PAP and AR antigens are provided on the same nucleic acid. In some embodiments, PAP and AR antigens are provided in two nucleic acids. In some embodiments, PAP and AR antigens are provided in two nucleic acids in the same composition. In some embodiments, PAP and AR antigens are provided in two nucleic acids in separate compositions.

In some embodiments, the PAP and/or AR genes are ligated into an expression vector that has been specifically optimized for polynucleotide vaccinations. Features of a suitable expression vector include, e.g., a transcriptional promoter, immunogenic epitopes, immunostimulatory sequences, and additional cistrons encoding immunoenhancing or immunomodulatory genes, with their own promoters, transcriptional terminators, bacterial origin of replication, and antibiotic resistance genes, as well known to those skilled in the art. Optionally, in some embodiments the vector contains internal ribosome entry sites (IRES) for the expression of polycistronic mRNA.

In some embodiments of the technology, a gene encoding a PAP and/or AR protein is directly linked to a transcriptional promoter. In some embodiments, a tissue-specific promoter or enhancer (e.g., the muscle creatine kinase (MCK) enhancer element) finds use to limit expression of the polynucleotide to a particular tissue type. For example, myocytes are terminally differentiated cells that do not divide. Integration of foreign DNA into chromosomes appears to be promoted by both cell division and protein synthesis. Thus, limiting protein expression to non-dividing cells such as myocytes may be preferable. In addition, in some embodiments a PSA promoter is used to limit expression of the protein to prostate tissue. In some embodiments, tissue-specific or cell-specific promoters are used to target the expression of the protein to antigen-presenting cells. For example, in some embodiments an alpha-fetoprotein (AFP) promoter (see e.g. Peyton et al. 2000, Proc. Natl. Acad. Sci., USA. 97:10890-10894) is used to limit expression to liver tissues. However, use of the CMV promoter is adequate for achieving expression in many tissues into which the plasmid DNA vaccine is introduced.

In various embodiments, suitable vectors include any plasmid DNA construct encoding a PAP and/or AR antigen or a functional equivalent or derivative thereof, operatively linked to a eukaryotic promoter. Examples of such vectors include the pCMV series of expression vectors, commercially available from Stratagene (La Jolla, Calif.); or the pCDNA or pREP series of expression vectors by Invitrogen Corporation (Carlsbad, Calif.).

There are many embodiments of the instant invention that those skilled in the art can appreciate from the specification. Thus, in the various embodiments different transcriptional promoters, terminators, and other transcriptional regulatory elements are used. Examples of other eukaryotic transcription promoters include the Rous sarcoma virus (RSV) promoter, the simian virus 40 (SV40) promoter, the human elongation factor-1 alpha (EF-1 alpha) promoter, and the human ubiquitin C (UbC) promoter.

In some embodiments, “naked” plasmid DNA expressing a transgene finds use, e.g., in some embodiments the naked plasmid DNA is directly injected intradermally or intramuscularly, taken up, and expressed (see e.g. Wolff et al., 1990, Science 247:1465-8). The efficiency of this approach may be low, with only a small percentage of myocytes being directly transformed in vivo, and within only a limited area of muscle tissue targeted by this directed delivery. Various alternative approaches yielding a higher efficiency gene delivery method are known (see e.g. Acsadi et al., 1991, New Biol. 3:71-81; Wolff et. al., 1991, Biotechniques 11:474-85; Budker et. al., 1996, Nat. Biotechnol. 14:760-4; Davis et al., 1993, Hum. Gene Ther. 4:151-9; Danko et al., 1994, Gene Ther. 1:114-21; Manthorpe et al., 1993, Hum. Gene Ther. 4:419-31).

In some embodiments, the DNA vaccine is pTVG-HP (e.g., pTVG4 vector containing cDNA for human PAP). pTVG-HP is a plasmid DNA, produced in E. coli, that encodes the cDNA for human prostatic acid phosphatase (PAP). In particular, the pTVG-HP plasmid was constructed from the plasmid vector pNGVL3 (e.g., as obtained from the National Gene Vector Laboratory at the University of Michigan). This vector, similar to the pCDNA3.1 expression vector, drives transcription from the CMV promoter, but also includes the CMV intron A sequence to enhance protein expression (Lee et al., 1997, Mol. Cells 7:495-501). The vector also contains a multi-cloning site, and does not express a eukaryotic antibiotic resistance gene, such that the only protein expression expected in a eukaryotic system is the one driven from the CMV promoter, unlike the pCDNA vector. To this vector has been added 2 copies of a 36-bp immunostimulatory (ISS) fragment containing the 5′-GTCGTT-3′ motif previously identified (Hartmann et al., 2000, J. Immunol. 164:1617-24) (e.g., a polynucleotide comprising a TpC dinucleotide at the 5′ end followed by three 6-mer CpG motifs (5′-GTCGTT-3′) separated by TpT dinucleotides), to create the vector pTVG4. The coding sequence for human PAP was cloned into this vector, and expression of PAP was confirmed by in vitro expression studies. This construct is named pTVG-HP. Thus, in some embodiments the DNA vaccine comprises CpG immunostimulatory sequences. In some embodiments, the immunostimulatory sequence is TCG TCG TTT TGT CGT TTT GTC GTT (SEQ ID NO: 4).

In some embodiments, vaccine comprise GM-CSF. GM-CSF (Leukine®, Sargramostim) is a vaccine adjuvant. In particular, GM-CSF is a growth factor that supports the survival, clonal expansion and differentiation of hematopoietic progenitor cells including dendritic antigen presenting cells. GM-CSF has been shown to be safe and serve as an effective adjuvant for the induction of antibody and T-cell responses to the immunized antigen [58, 59]. The use of GM-CSF is associated with little toxicity [60, 61, 62]. GM-CSF is a sterile, white, preservative-free, lyophilized powder supplied in 250 μg-dose vials. Recombinant human GM-CSF (rhGM-CSF), when administered intravenously or subcutaneously is generally well tolerated at doses ranging from 50 to 500 μg/m2/day.

In specific embodiments of the technology, vials are thawed and the plasmid DNA vaccine is used to reconstitute the GM-CSF. For example, for each DNA immunization, 0.6 mL of 0.2 mg/mL pTVG-HP is withdrawn and used to reconstitute 250 μg GM-CSF. 0.25 mL is then drawn into each of two tuberculin syringes. This effectively provides a 100-μg dose of DNA and 208 μg GM-CSF.

### II. PD-1 Blockage

A major mechanism by which tumors avoid immune detection is by expressing PD-L1 or PD-L2, which are ligands for the T-cell receptor PD-1. Activation of PD-1 by PD-L1 or PD-L2 decreases T-cell function and increases immune tolerance. There is currently great enthusiasm to develop PD/PD-L (e.g., PD-1 and/or PD-L1) inhibitors given the relative paucity of adverse events observed with these agents in clinical trials and long-term disease response observed in some instances in early phase clinical trials. Targeting PD-1, in particular, should be a universal therapy, as it targets the T-cell compartment rather than the tumor directly. However, clinical trial experience to date suggests that patients with some solid tumor types (notably renal cell cancer, melanoma, and non-small cell lung cancer) experience more benefit than patients with other histologies, including prostate cancer [18, 19]. Differences in the T-cells of responding and non-responding patients may be the basis of this disparity. In particular, higher frequencies of tumor-infiltrating lymphocytes (TIL) are typically observed in patients with renal cell cancer and melanoma than prostate cancer. In addition, early phase clinical trials using PD-1 or PD-L1 have identified that the expression of at least one of the ligands for PD-1 (PD-L1) on the target tumor cell by biopsy is associated with clinical response to therapy [18]. This is expected, given that tissue-infiltrating T cells can induce the expression of PD-L1 via the expression of IFNγ, and ligand binding of PD-1 leads to decrease in T-cell effector function. It has been demonstrated that prostate cancers can express PD-L1, and can have infiltrating PD-1-expressing T cells [20]. Taken together, these results indicate that the efficacy of anti-tumor immunotherapy would be increased for prostate cancer by combining agents able to increase the number of tumor-specific T cells, such as through vaccination, and by PD blockade and/or by PD-L blockade (e.g., PD-1 and/or PD-L1 blockade), e.g., by a PD inhibitor and/or by a PD-L inhibitor (e.g., a PD-1 inhibitor (e.g., an anti-PD-1 antibody) and/or a PD-L1 inhibitor (e.g., an anti-PD-L1 antibody)).

Given the anti-tumor responses observed in early phase clinical trials with antibodies targeting either PD-1 or PD-L1, several pharmaceutical companies have been developing related agents. Currently, one agent has been approved as a therapy, pembrolizumab (KEYTRUDA, Merck). Specifically, pembrolizumab was approved in September 2014 for the treatment of ipilimumab-refractory advanced melanoma as a “breakthrough” therapy on the basis of an open-label, international, multicenter expansion cohort of a phase I trial of patients with advanced (metastatic) melanoma whose disease had progressed following treatment with ipilimumab. In that trial, 173 patients received pembrolizumab at one of two doses (2 mg/kg or 10 mg/kg) at 3-week intervals until disease progression or intolerable toxicity. An overall response rate of 26% was observed, irrespective of dose. Grade 3 fatigue was the only drug-related grade 3 or 4 adverse event reported in more than one patient. Given these findings, pembrolizumab is currently FDA approved for the treatment of patients with ipilimumab-refractory melanoma, dosed at 2 mg/kg intravenously every 3 weeks until disease progression or intolerable adverse effects. Of note, however, earlier phase clinical trials have suggested that treatment can lead to prolonged responses even after discontinuing treatment.

In some embodiments, the PD-1 pathway inhibitor is a monoclonal antibody. In some embodiments, the monoclonal antibody is pembrolizumab (marketed under the trade name “Keytruda®”). Pembrolizumab is a human programmed death receptor-1 (PD-1)-blocking antibody indicated for the treatment of patients with unresectable or metastatic melanoma and disease progression following ipilimumab and, if BRAF V600 mutation positive, a BRAF inhibitor. Pembrolizumab is available in single-use vials, consisting of 100 mg lyophilized powder for injection and is preferably administered at a 200 mg fixed dose every three weeks. It is prepared by addition of 4.0 mL of sterile water for injection, USP, to the vial to prepare a 25 mg/mL solution. In some embodiments, the contents of two vials are transferred to an IV bag containing 0.9% sodium chloride injection, USP such that the final concentration of the diluted solution is between 1 mg/mL and 10 mg/mL. Accordingly, in some embodiments it is administered as an intravenous infusion, e.g., over 30 minutes using an IV line containing a sterile, non-pyrogenic, low-protein binding 0.2 μm to 5 μm in-line or add-on filter.

In some embodiments, the monoclonal antibody is nivolumab (marketed under the trade name “Opdivo®”). Nivolumab is a human IgG4 anti-PD-1 monoclonal antibody that acts as an immunomodulator by blocking ligand activation of the programmed cell death 1 (PD-1) receptor on activated T cells. In particular, nivolumab acts by blocking a negative regulator of T-cell activation and response, thus allowing the immune system to attack the tumor. That is, nivolumab blocks PD-L1 from binding to PD-1, allowing the T cell to function in tumor attack.

The present invention also contemplates the use of other PD-1 antagonists in the methods and kits of the present invention, including, but not limited to: BMS-936559 (Bristol-Myers Squibb); MEDI0680 (MedImmune/AstraZeneca); MEDI4736 (MedImmune/AstraZeneca); MPDL3280A (Genentech/Roche), MSB0010718C (EMD Serono); and Pidilizumab (CureTech).

### III. Androgen Depravation Therapy

In some embodiments, prostate cancer therapies include androgen depravation therapy (ADT). The synthesis of testosterone is mediated by a chain of processes that start in the brain. When the body detects a low level of testosterone, the hypothalamus starts to produce LHRH, a hormone which, once is received by the pituitary gland activates the synthesis of LH (Luteinizing hormone). LH travels to the testicles where it induces the formation of testosterone. There are two methods of androgen deprivation therapy based on drugs. One works preventing the pituitary gland from releasing LH and the other one blocks the body's ability to use androgens.

There are two different medicines, LHRH agonists and antagonists, which both lower the amount of testosterone made by the testicles. They work inhibiting the formation of LH in the pituitary gland. The LHRH agonists produce a sudden increase in levels of testosterone followed by a huge falling, process called flare, whereas LHRH antagonists decrease directly the amount of testosterone. Examples of LHRH agonist and antagonist active substances include, but are not limited to, leuprolide, goserelin, triptorelin, histrelin and degarelix. In some embodiments, these drugs are injected under the skin achieving the same result as surgical castration.

In some embodiments, anti-androgen therapy (e.g., androgen receptor antagonists) is utilized. Adrenal glands were discovered as another center of androgen production even after a castration process. Therefore a complementary treatment was developed that uses antiandrogens to block the body's ability to use any androgens. Prostate cells contain an Androgen Receptor (AR), that when stimulated by androgens like testosterone, promotes growth and maintains prostatic differentiation. These pro-growth signals, however, can be problematic when they occur in a cancer cell. Antiandrogens can enter cells and prevent the binding of testosterone to the receptor proteins, due to their higher affinity for the androgen receptor.

Examples of androgen receptor antagonists include, but are not limited to, cyproterone acetate, flutamide, nilutamide, bicalutamide, and enzalutamide, and apalutamide, which are all administered in oral pill form and. Additional androgen receptor antagonists include, but are not limited to, agents that target testosterone synthesis (e.g., abiraterone acetate and seviteronel) or AR nuclear translocation (e.g., enzalutamide, apalutamide, and darolutamide), as well as combined therapies (e.g., galeterone).

In some embodiments, the androgen receptor antagonist is enzalutamide or apalutamide.

### IV. Treatment Methods

As described above, provided herein are combination therapies for prostate cancer treatment. In some embodiments, therapies comprise one or more DNA vaccines (e.g., DNA vaccines that target PAP and/or AR) in combination with PD-1 blockade and androgen receptor antagonist.

In some embodiments, the DNA vaccine (e.g., DNA vaccines that target PAP and/or AR) is administered concurrently with the PD pathway inhibitor and AR antagonist as discussed herein (see, e.g., dosing schedules described in detail herein). In some embodiments, the DNA vaccine (e.g., DNA vaccines that target PAP and/or AR) is administered prior to, concurrently, or after treatment with the PD pathway inhibitor (see, e.g., dosing schedules described in detail herein).

In some embodiments, the DNA vaccine and PD-1 pathway inhibitor are administered concurrently for 1, 2, 3, 4, 5, 6, 7, 8, 9, 10, 11, 12, or more weeks followed by a period in which the DNA vaccine, the PD-1 pathway inhibitor and the AR antagonist are administered concurrently for 1, 2, 3, 4, 5, 6, 7, 8, 9, 10, 11, 12, or more weeks, followed by a period in which the DNA vaccine, the PD-1 pathway inhibitor and the AR antagonist are not administered (e.g., 1, 4, 6, 8, 10, 12, 14, 16, 18, 24 or more weeks).

In some embodiments, the AR antagonist is administered in intervals of multiple weeks (e.g., one or more times daily for 1, 2, 3, 4, 5, 6, 7, 8, 9, 10, 11, 12, or more weeks) concurrently with the DNA vaccine and PD-1 pathway inhibitor, followed by a period in which the AR antagonist is not administered (e.g., 1, 4, 6, 10, 12, 16, 18, 24 or more weeks). In some embodiments, periods of AR antagonist treatment are administered during long term treatment with DNA vaccine and PD-1 pathway inhibitor.

Thus, some embodiments of the technology relate to administering pembrolizumab or nivolumab in combination (e.g., concurrently or sequentially) with DNA vaccines and AR antagonist in cancer patients (e.g., patients with non-metastatic, hormone-sensitive, biochemically-recurrent (PSA-recurrent) prostate cancer or patients with metastatic, castration-resistant prostate cancer).

In some embodiments, patients are tested for response to the combination therapy before and/or after administration of the DNA vaccine. In some embodiments, patients are tested before and/or after administration of the PD pathway inhibitor (e.g., pembrolizumab or nivolumab). In some embodiments, testing comprises, e.g., imaging methods (e.g., radiographic methods, bone imaging), measuring anti-tumor response rates (objective response rate and/or PSA response rate, using PCWG2 criteria), measuring the magnitude of PAP- or AR-specific T-cell responses, measuring PD-1 expression on circulating T cells, measuring PD-L1 expression on circulating epithelial cells (CEC) and/or on tumor biopsies, measuring tumor growth rates, measuring the amounts of PAP-specific antibodies, measuring the amounts of prostate-associated antigens (e.g., PSA and/or PAP).

In some embodiments, biomarkers are monitored, e.g., to follow the course of treatment and/or as predictors of the efficacy of treatment. Exemplary biomarkers include PD-L1 expression on CEC or tumor biopsies, expression of other regulatory molecules on tumor-specific T cells (e.g. TIM3, BTLA, and LAGS) or tumor cells (e.g. HVEM, phosphatidyl serine, PD-L2), and PD-1-regulated antigen-specific T cells (e.g., identified by trans vivo DTH testing).

It is generally contemplated that the DNA vaccine, the AR antagonist, and the PD-1 inhibitor are formulated for administration to a mammal, and especially to a human with a condition that is responsive to the administration of such compounds (e.g., a human subject having a prostate cancer). Therefore, where contemplated compounds are administered in a pharmacological composition, it is contemplated that the contemplated compounds are formulated in admixture with a pharmaceutically acceptable carrier. For example, contemplated compounds can be administered orally as pharmacologically acceptable salts, or intravenously in a physiological saline solution (e.g., buffered to a pH of about 7.2 to 7.5). Conventional buffers such as phosphates, bicarbonates, or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound to manage the pharmacokinetics of the present compounds for maximum beneficial effect in a patient.

In certain pharmaceutical dosage forms, prodrug forms of contemplated compounds may be formed for various purposes, including reduction of toxicity, increasing the organ or target cell specificity, etc. Among various prodrug forms, acylated (acetylated or other) derivatives, pyridine esters, and various salt forms of the present compounds are preferred. One of ordinary skill in the art will recognize how to readily modify the present compounds to prodrug forms to facilitate delivery of active compounds to a target site within the host organism or patient. One of ordinary skill in the art will also take advantage of favorable pharmacokinetic parameters of the prodrug forms, where applicable, in delivering the present compounds to a targeted site within the host organism or patient to maximize the intended effect of the compound. Similarly, it should be appreciated that contemplated compounds may also be metabolized to their biologically active form, and all metabolites of the compounds herein are therefore specifically contemplated. In addition, contemplated compounds (and combinations thereof) may be administered in combination with yet further agents.

With respect to administration to a subject, it is contemplated that the compounds be administered in a pharmaceutically effective amount. One of ordinary skill in the art recognizes that a pharmaceutically effective amount varies depending on the therapeutic agent used, the subject's age, condition, and sex, and on the extent of the disease in the subject. Generally, the dosage should not be so large as to cause adverse side effects, such as hematological problems, pulmonary problems, colitis, hepatitis, nephritis, hypophisitis, impaired thyroid function, and the like. The dosage can also be adjusted by the individual physician to achieve the desired therapeutic goal. Thus it is contemplated that in men with low tumor burden disease (e.g., men with non-metastatic, or micro-metastatic hormone-sensitive, biochemically-recurrent prostate cancer) they might be dosed with ½ or ⅓ or ¼ of the standard dose of a PD-1 inhibitor to elicit both effective anti-tumor response and reduced toxicity and side effects typically attributed to PD-1 inhibitors at standard does used in advanced or metastatic cancers.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to maximize efficacy but will depend on such factors as weight, diet, concurrent medication, and other factors that those skilled in the art will recognize.

Pharmaceutical compositions preferably comprise one or more compounds of the present technology associated with one or more pharmaceutically acceptable carriers, diluents, or excipients. Pharmaceutically acceptable carriers are known in the art such as those described in, for example, Remingtons Pharmaceutical Sciences, Mack Publishing Co. (A. R. Gennaro edit. 1985), explicitly incorporated herein by reference for all purposes.

Accordingly, in some embodiments, the immunotherapeutic agent is formulated as a tablet, a capsule, a time release tablet, a time release capsule; a time release pellet; a slow release tablet, a slow release capsule; a slow release pellet; a fast release tablet, a fast release capsule; a fast release pellet; a sublingual tablet; a gel capsule; a microencapsulation; a transdermal delivery formulation; a transdermal gel; a transdermal patch; a transdermal dissolvable microneedle formulation (e.g., provided in a patch); a sterile solution; a sterile solution prepared for use as an intramuscular, intradermal, or subcutaneous injection, for use as a direct injection into a targeted site, or for intravenous administration; a solution prepared for rectal administration; a solution prepared for administration through a gastric feeding tube or duodenal feeding tube; a suppository for rectal administration; a liquid for oral consumption prepared as a solution or an elixir; a topical cream; a gel; a lotion; a tincture; a syrup; an emulsion; or a suspension.

In some embodiments, the time release formulation is a sustained-release, sustained-action, extended-release, controlled-release, modified release, or continuous-release mechanism, e.g., the composition is formulated to dissolve quickly, slowly, or at any appropriate rate of release of the compound over time.

In some embodiments, the compositions are formulated so that the active ingredient is embedded in a matrix of an insoluble substance (e.g., various acrylics, chitin) such that the dissolving compound finds its way out through the holes in the matrix, e.g., by diffusion. In some embodiments, the formulation is enclosed in a polymer-based tablet with a laser-drilled hole on one side and a porous membrane on the other side. Stomach acids push through the porous membrane, thereby pushing the drug out through the laser-drilled hole. In time, the entire drug dose releases into the system while the polymer container remains intact, to be excreted later through normal digestion. In some sustained-release formulations, the compound dissolves into the matrix and the matrix physically swells to form a gel, allowing the compound to exit through the gel's outer surface. In some embodiments, the formulations are in a micro-encapsulated form, e.g., which is used in some embodiments to produce a complex dissolution profile. For example, by coating the compound around an inert core and layering it with insoluble substances to form a microsphere, some embodiments provide more consistent and replicable dissolution rates in a convenient format that is combined in particular embodiments with other controlled (e.g., instant) release pharmaceutical ingredients, e.g., to provide a multipart gel capsule.

In some embodiments, the pharmaceutical preparations and/or formulations of the technology are provided in particles. “Particles” as used herein means nano- or microparticles (or in some instances larger) that can consist in whole or in part of the compounds as described herein. The particles may contain the preparations and/or formulations in a core surrounded by a coating, including, but not limited to, an enteric coating. The preparations and/or formulations also may be dispersed throughout the particles. The preparations and/or formulations also may be adsorbed into the particles. The particles may be of any order release kinetics, including zero order release, first order release, second order release, delayed release, sustained release, immediate release, and any combination thereof, etc. The particle may include, in addition to the preparations and/or formulations, any of those materials routinely used in the art of pharmacy and medicine, including, but not limited to, erodible, nonerodible, biodegradable, or nonbiodegradable materials or combinations thereof. The particles may be microcapsules which contain the formulation in a solution or in a semi-solid state. The particles may be of virtually any shape.

Both non-biodegradable and biodegradable polymeric materials can be used in the manufacture of particles for delivering the preparations and/or formulations. Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26: 581-587, the teachings of which are incorporated herein by reference. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly (isobutyl methacrylate), poly(hexylmethacrylate), poly(isodecyl methacrylate), poly(lauryl methacrylate), poly(phenylmethacrylate), poly(methyl acrylate), poly(isopropyl acrylate), poly(isobutyl acrylate), and poly(octadecyl acrylate).

The technology also provides methods for preparing stable pharmaceutical preparations containing aqueous solutions of the compounds or salts thereof to inhibit formation of degradation products. A solution is provided that contains the compound or salts thereof and at least one inhibiting agent. The solution is processed under at least one sterilization technique prior to and/or after terminal filling the solution in the sealable container to form a stable pharmaceutical preparation. The present formulations may be prepared by various methods known in the art so long as the formulation is substantially homogenous, e.g., the pharmaceutical is distributed substantially uniformly within the formulation. Such uniform distribution facilitates control over drug release from the formulation.

In some embodiments, the compound is formulated with a buffering agent. The buffering agent may be any pharmaceutically acceptable buffering agent. Buffer systems include citrate buffers, acetate buffers, borate buffers, and phosphate buffers. Examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

In some embodiments, the compound is formulated with a chelating agent. The chelating agent may be any pharmaceutically acceptable chelating agent. Chelating agents include ethylenediaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid and derivatives thereof. Citric acid also is known as citric acid monohydrate. Derivatives of citric acid include anhydrous citric acid and trisodiumcitrate-dihydrate. Still other chelating agents include niacinamide and derivatives thereof and sodium desoxycholate and derivatives thereof.

In some embodiments, the compound is formulated with an antioxidant. The antioxidant may be any pharmaceutically acceptable antioxidant. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, and sodium sulfite. Such materials are typically added in ranges from 0.01 to 2.0%.

In some embodiments, the compound is formulated with a cryoprotectant. The cryoprotecting agent may be any pharmaceutically acceptable cryoprotecting agent. Common cryoprotecting agents include histidine, polyethylene glycol, polyvinyl pyrrolidine, lactose, sucrose, mannitol, and polyols.

In some embodiments, the compound is formulated with an isotonicity agent. The isotonicity agent can be any pharmaceutically acceptable isotonicity agent. This term is used in the art interchangeably with isoosmotic agent, and is known as a compound which is added to the pharmaceutical preparation to increase the osmotic pressure, e.g., in some embodiments to that of 0.9% sodium chloride solution, which is iso-osmotic with human extracellular fluids, such as plasma. Preferred isotonicity agents are sodium chloride, mannitol, sorbitol, lactose, dextrose and glycerol.

The pharmaceutical preparation may optionally comprise a preservative. Common preservatives include those selected from the group consisting of chlorobutanol, parabens, thimerosol, benzyl alcohol, and phenol. Suitable preservatives include but are not limited to: chlorobutanol (0.3-0.9% w/v), parabens (0.01-5.0%), thimerosal (0.004-0.2%), benzyl alcohol (0.5-5%), phenol (0.1-1.0%), and the like.

In some embodiments, the compound is formulated with a humectant to provide a pleasant mouth-feel in oral applications. Humectants known in the art include cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

### Methods of Measurement and Assays

In various embodiments, data are collected using various techniques and observables, e.g., to measure a baseline for a subject and/or to monitor the efficacy of a treatment. For instance, some embodiments comprise imaging-based evaluation of a subject. In some particular embodiments, imaging techniques comprise computed tomography (CT). In some particular embodiments, imaging techniques comprise magnetic resonance imaging (MRI). In some embodiments, CT and/or MRI provide accurate and reproducible methods for measuring target lesions. In some embodiments, CT and MRI are performed with contiguous cuts of 10 mm or less in slice thickness. In some embodiments, spiral CT is performed using a 5 mm contiguous reconstruction algorithm, e.g., for tumors of the chest, abdomen, and pelvis.

In some embodiments, a tumor marker is measured. For example, in some embodiments, PSA is measured. In some embodiments, PSA values are collected for separate reporting of PSA kinetics. In some embodiments, a value of PSA that declines to <0.2 ng/mL for a subject indicates a complete PSA response. In some embodiments, serum PAP and/or AR is measured. In some embodiments, serum concentrations of PAP and/or PSA stabilize after vaccination and/or decline after vaccination and provide a measurement of vaccine efficacy. In some embodiments, the ratio of serum PSA to serum PAP or AR is calculated and provides a measure of vaccine efficacy. Without being bound by theory and with an understanding that an understanding of the mechanism or theory is not required to practice the technology, in some embodiments the PSA:PAP ratio increases with therapy because therapy selectively depletes PAP-producing cells relative to PSA-producing cells, thus causing PAP concentrations to fall faster than PSA concentrations.

In some embodiments, a clinical examination is performed on a subject. In some embodiments, a clinically detected lesion is considered measurable when it is superficial (e.g., skin nodules and palpable lymph nodes). In some embodiments, skin lesions are documented by color photography, including a ruler to estimate size of the lesion.

### Histopathology Evaluation

In some embodiments, tissue biopsies are obtained from metastatic lesions (e.g., the same lesion per patient) prior to treatment and after treatment (e.g., from 1 to 24 weeks after treatment is initiated, e.g., during week 1, 2, 3, 4, 5, 6, 7, 8, 9, 10, 11, 12, 13, 14, 14, 16, 17, 18, 19, 20, 21, 22, 23, or 24). In some embodiments, tests are performed to evaluate how immunization affects PD-L1 expression in the tumor. Without being bound by theory and acknowledging that an understanding of the mechanism is not required to practice the technology, it is contemplated that immunization affects PD-L1 expression in the tumor by eliciting tumor antigen-specific T cells secreting IFNγ. Furthermore, tests are performed to assess whether concurrent treatment with anti-PD-1 mAb leads to an increase in infiltration of CD8+ T cells and whether treatment increases expression of other T-cell regulatory ligands on T cells (PD-1, CTLA-4, TIM-3, BTLA, LAG-3) or tumors (e.g. HVEM, phosphatidyl serine, PD-L2). Consequently, biopsy specimens obtained pre-treatment and after initiation of treatment are stained with antibodies specific for CD3, CD4, CD8, FoxP3, PD-1, CTLA-4, TIM3, BTLA, LAG-3, PD-L1, PD-L2, phosphatidyl serine, and/or HVEM, and/or other markers. Staining and quantification are reviewed by a pathologist blinded to the treatment groups to determine CD8+ T cells per field, CD4+FoxP3+ (Treg):CD8+ T cell ratio, PD-L1 expression (e.g., as indicated by at least 5 PD-L1 positive cells per field), and to compare these parameters measured before and after treatment to identify any of these measured or calculated parameters that changed as a result of treatment.

### Circulating Tumor Cell (CTC) Evaluation

In some embodiments, CTC are enumerated and characterized, e.g., in some embodiments, at the same time points as for immune evaluation (e.g., pre-treatment, after 1 to 6 weeks (e.g., after 1, 2, 3, 4, 5, or 6 weeks), and at intervals for up to one year (e.g., monthly, quarterly) using flow cytometry. For example, in some embodiments PBMC obtained at these time points are stained, e.g., with fluorochrome-labeled antibodies specific for at least one or more of CD45, EpCAM, PD-1, PD-L1, CTLA-4, and DAPI. CTC are defined as CD45-EpCAM+DAPI− cells, and the percentage of these events among all live cellular events are determined at the different time points. The percentages of CTC expressing PD-1, PD-L1, AR, or CTLA-4 are also determined. Results are reported from the different time points and general trends are assessed. In some embodiments, results quantitative and in some embodiments, results are qualitative (e.g., in some embodiments, obtaining multiple replicates to determine standard deviation are not feasible). The technology encompasses other methods of CTC capture and enumeration.

### Subjects

In some embodiments, the combination therapy is administered to a subject. For example, in some embodiments, the subject is a cancer patient, e.g., a prostate cancer patient, e.g., a patient having non-metastatic, hormone-sensitive, biochemically-recurrent prostate cancer or a patient having metastatic, castrate-resistant prostate cancer. In some embodiments, the subject is an adult (e.g., is aged 18 years or more). In some embodiments, the subject has prostate cancer that has been confirmed by histology. In some embodiments, the subject has metastatic disease, e.g., the presence of soft tissue and/or bone metastases (e.g., as detected by imaging (CT (e.g., of abdomen/pelvis), bone scintigraphy, etc.)). In some embodiments, the subject has castrate-resistant disease, e.g., in some embodiments, subjects have received androgen deprivation treatment (e.g., surgical castration, GnRH analogue, or antagonist treatment). In some embodiments, subjects receive a GnRH analogue or antagonist during treatment with the combination therapy (e.g., DNA vaccine, AR antagonist, and PD-1 inhibitor) described herein. In some embodiments, subjects have been treated previously with a nonsteroidal antiandrogen; in some embodiments, subjects have not been treated previously with a nonsteroidal antiandrogen. In some embodiments in which subjects have been previously treated with an antiandrogen, the subjects have discontinued use of anti-androgen for at least 4 weeks (for flutamide) or 6 weeks (for bicalutamide or nilutamide) prior to treatment with the combination therapy described herein. Moreover, in some embodiments, PSA is monitored in subjects, e.g., for subjects who demonstrated an anti-androgen withdrawal response (e.g., a >25% decline in PSA within 4-6 week of stopping a nonsteroidal antiandrogen), the combination therapy described herein is administered when the subject PSA rises above the nadir observed after antiandrogen withdrawal. In some embodiments, the castration level of testosterone is <50 ng/dL within 6 weeks the beginning of treatment.

In some embodiments, subjects have been previously treated with abiraterone or enzalutamide and, in some embodiments, subjects have been off (e.g., discontinued) prior corticosteroid treatment for at least 3 months. In some embodiments, subjects have an ECOG performance status of 0, 1, or 2. In some embodiments, subjects have adequate hematologic, renal, and liver function (e.g., WBC>2000/mm³; ANC>1000/mm³; HgB>9.0 gm/dL; platelets >100,000/mm³; creatinine <2.0 mg/dL; and/or AST, ALT <2.5× institutional upper limit of normal). In some embodiments, subjects have no history of HIV 1 and 2, HTLV-1, or active Hepatitis B or Hepatitis C. In some embodiments, subjects have not had other treatments for at least 4 weeks and have recovered (to <Grade 2) from acute toxicity attributed to prior treatment. In some embodiments, subjects have had a biopsy.

In some embodiments, the level of PD-1, PD-L1, and/or AR in the subjected are measured to assess or modify treatment. In some embodiments, measuring the levels and administering the DNA vaccine are performed in any order and frequency without limitation, e.g., measure/administer, administer/measure, measure/administer/measure, administer/measure/administer, measure/administer/measure/administer, measure/administer/measure/administer/measure, measure/administer/measure/measure/administer/administer/administer/measure, administer/administer/measure/administer, measure/administer/administer/measure/administer/administer, etc.

In some embodiments, treatment schedules or dosages are altered in response to the measuring (e.g., to obtain a target level of PD-1, PD-L1, and/or AR).

### Administration, Treatments, Dosages, and Dosing Schedules

In some embodiments, the technology relates to methods of administering a DNA vaccine and a PD-1 inhibitor to a subject in combination with an AR antagonist. In some embodiments, the AR antagonist is administered at a different dosing schedule from the DNA vaccine and PD-1 inhibitor.

In some embodiments, the technology relates to methods of administering a DNA vaccine and a PD-1 inhibitor to a subject. The methods comprise the general steps of administering a DNA vaccine and a PD-1 inhibitor to a subject according to the technology. In some embodiments, a DNA vaccine and/or a PD-1 inhibitor, a derivative thereof, or a pharmaceutically acceptable salt thereof, is administered in a pharmaceutically effective amount. In some embodiments, a DNA vaccine and/or a PD-1 inhibitor, a derivative thereof, or a pharmaceutically acceptable salt thereof, is administered in a therapeutically effective dose.

The dosage amount and frequency are selected to create an effective level of the compound without substantially harmful effects. When administered orally, intradermally, transdermally, or intravenously, the dosage of the DNA vaccine and/or a PD-1 inhibitor will generally range from 0.001 to 10,000 mg/kg/day or dose (e.g., 0.01 to 1000 mg/kg/day or dose; 0.1 to 100 mg/kg/day or dose).

Methods of administering a pharmaceutically effective amount include, without limitation, administration in parenteral, oral, intraperitoneal, intranasal, topical, sublingual, rectal, and vaginal forms. Parenteral routes of administration include, for example, subcutaneous, intravenous, intramuscular, intrastemal injection, intravenous, intradermal, transdermal and infusion routes. In some embodiments, the compound, a derivative thereof, or a pharmaceutically acceptable salt thereof, is administered orally.

In some embodiments, a single dose of a compound or a related compound is administered to a subject. In other embodiments, multiple doses are administered over two or more time points, separated by hours, days, weeks, etc. In some embodiments, compounds are administered over a long period of time (e.g., chronically), for example, for a period of weeks, months, or years (e.g., 1, 2, 3, 4, 5, 6, 7, 8, 9, 10, 11, 12, or more weeks, months, or years). In such embodiments, compounds may be taken on a regular scheduled basis (e.g., daily, weekly, fortnightly, etc.) for the duration of the extended period.

The technology also relates to methods of treating a subject with a DNA vaccine and a PD-1 inhibitor. According to another aspect of the technology, a method is provided for treating a subject in need of such treatment with an effective amount of a DNA vaccine and/or a PD-1 inhibitor or one or more salts thereof. The method involves administering to the subject an effective amount of a DNA vaccine and/or a PD-1 inhibitor or a salt thereof in any one of the pharmaceutical preparations described above, detailed herein, and/or set forth in the claims. The subject can be any subject in need of such treatment. In the foregoing description, the technology is in connection with a compound or salts thereof. Such salts include, but are not limited to, bromide salts, chloride salts, iodide salts, carbonate salts, and sulfate salts. It should be understood, however, that the compound is a member of a class of compounds and the technology is intended to embrace pharmaceutical preparations, methods, and kits containing related derivatives within this class. Another aspect of the technology then embraces the foregoing summary but read in each aspect as if any such derivative is substituted wherever “compound” appears.

In some embodiments, a subject is tested to assess the presence, the absence, or the level of a malady and/or a condition such as prostate cancer. Such testing is performed, e.g., by assaying or measuring a biomarker, a metabolite, a physical symptom, an indication, etc., to determine the risk of or the presence of the malady or condition. In some embodiments, the subject is treated with a DNA vaccine and/or a PD-1 inhibitor based on the outcome of the test. In some embodiments, a subject is treated with a DNA vaccine and/or a PD-1 inhibitor, a sample is obtained, and the level of detectable agent is measured, and then the subject is treated again with a DNA vaccine and/or a PD-1 inhibitor based on the level of detectable agent that was measured. In some embodiments, a subject is treated with a DNA vaccine and/or a PD-1 inhibitor, a sample is obtained and the level of detectable agent is measured, the subject is treated again with a DNA vaccine and/or a PD-1 inhibitor based on the level of detectable agent that was measured, and then another sample is obtained and the level of detectable agent is measured. In some embodiments, other tests (e.g., not based on measuring the level of detectable agent) are also used at various stages, e.g., before the initial treatment with a DNA vaccine and/or a PD-1 inhibitor as a guide for the initial dose. In some embodiments, a subsequent treatment with a DNA vaccine and/or a PD-1 inhibitor is adjusted based on a test result, e.g., the dosage amount, dosage schedule, identity of the drug, etc. is changed.

In some embodiments, a patient is tested, treated, and then tested again to monitor the response to therapy and/or change the therapy. In some embodiments, cycles of testing and treatment may occur without limitation to the pattern of testing and treating, the periodicity, or the duration of the interval between each testing and treatment phase. As such, the technology contemplates various combinations of testing and treating without limitation, e.g., test/treat, treat/test, test/treat/test, treat/test/treat, test/treat/test/treat, test/treat/test/treat/test, test/treat/test/test/treat/treat/treat/test, treat/treat/test/treat, test/treat/treat/test/treat/treat, etc.

The technology is not limited in the dosing and dosing schedule used to administer the DNA vaccine and the PD-1 inhibitor. For example, in some embodiments the DNA vaccine (e.g., pTVG-HP (e.g., 100 μg) with rhGM-CSF (e.g., 208 μg) or pTVG-HP (e.g., 100 ug) with GM-CSF (e.g. 208 ug) and pTVG-AR (e.g., 100 ug) with rhGM-CSF (e.g., 208 ug) administered as alternating doses is administered intradermally biweekly 6 times beginning at day 1 and the PD-1 inhibitor (e.g., pembrolizumab or nivolumab at a fixed dose of about 200 mg) is administered intravenously every 3 weeks 4 times, beginning at day 1. In some embodiments, the DNA vaccine (e.g., pTVG-HP (e.g., 100 μg) with rhGM-CSF (e.g., 208 μg)) is administered intradermally biweekly 6 times beginning at day 1 and the PD-1 inhibitor (e.g., pembrolizumab or nivolumab at a fixed dose of about 200 mg) is administered intravenously biweekly, every three weeks or every 4 weeks (e.g., from 3 to 6 times as appropriate) with the first dose being administered two weeks after the last pTVG-HP vaccination.

Thus, in some embodiments the DNA vaccine (e.g., pTVG-HP) is administered in an amount of approximately 100 μg (e.g., 10, 20, 30, 40, 50, 60, 70, 80, 90, 100, 110, 120, 130, 140, 150, 160, 170, 180, 190, 200, 300, 400, 500, 600, 700, 800, 900, or 1000 or more μg). In some embodiments, the DNA vaccine is administered in combination with an adjuvant, e.g., GM-CSF (e.g., rhGM-CSF), e.g., in an amount of approximately 200 μg (e.g., 100 to 500 μg, e.g., 100, 110, 120, 130, 140, 150, 160, 170, 180, 190, 200 (e.g., in some embodiments 208), 210, 220, 230, 240, 250, 260, 270, 280, 290, 300, 400, 500, 600, 700, 800, 900, or 1000 or more μg). As noted above, in some embodiments, the DNA vaccine is administered intradermally (e.g., on the deltoid area of the lateral arm). In some embodiments, the DNA vaccine is administered in a volume of approximately 0.25 mL (e.g., 100 to 500 μl, e.g., 100, 110, 120, 130, 140, 150, 160, 170, 180, 190, 200, 210, 220, 230, 240, 250, 260, 270, 280, 290, 300, 400, 500, 600, 700, 800, 900, or 1000 μl or more). In some embodiments, the volume of the DNA vaccine is administered at each of two adjacent sites. In some embodiments, the DNA vaccine is administered biweekly (e.g., approximately every two weeks, e.g., approximately every 14 days (e.g., 14±3 days (e.g., 11 to 17 days)). In some embodiments, the DNA vaccine is administered 6 times (e.g., 3 to 9 times, e.g., 3, 4, 5, 6, 7, 8, or 9 times).

Further, in some embodiments, the PD-1 pathway inhibitor (e.g., pembrolizumab or nivolumab) is administered at a dose of 2 mg/kg (e.g., 1 to 5 mg/kg, e.g., 1, 1.5, 2, 2.5, 3, 3.5, 4, 4.5, or 5 mg/kg) and most preferably at a fixed dose of about 200 mg. In some embodiments, the PD-1 pathway inhibitor is administered intravenously. In some embodiments, the PD-1 pathway inhibitor is administered (e.g., intravenously) over 30 minutes (e.g., from 15 minutes to 2 hours, e.g., for 15, 30, 45, 60, 75, 90, 105, or 120 minutes). In some embodiments, the PD-1 pathway inhibitor is administered every 3 weeks (e.g., every week, every 1.5 weeks, every 2 weeks, every 2.5 weeks, every 3 weeks (e.g., every 21±3 days (e.g., 18 to 228 days)), every 3.5 weeks, every 4 weeks, or every 5 weeks). In some embodiments, the PD-1 pathway inhibitor is administered 4 times (e.g., 2 to 9 times, e.g., 2, 3, 4, 5, 6, 7, 8, or 9 times). In some embodiments, the PD-1 pathway inhibitor is administered concurrently with the DNA vaccine, e.g., on Day 1 before, with, or after the first vaccination with the DNA vaccine. In some embodiments in which the PD-1 pathway inhibitor is administered concurrently with the DNA vaccine, the PD-1 pathway inhibitor is administered in a first composition and the DNA vaccine is administered in a second separate composition. In some embodiments in which the PD-1 pathway inhibitor is administered concurrently with the DNA vaccine, the PD-1 pathway inhibitor and the DNA vaccine are administered in the same composition. In another exemplary embodiment, the PD-1 pathway inhibitor is administered subsequently to the administration of the DNA vaccine, e.g., from 1, 2, 3, 4 or more weeks after the ultimate vaccination with the DNA vaccine.

In some embodiments, the technology comprises administering the DNA vaccine and the PD-1 pathway inhibitors according to a schedule. For example, in some embodiments a subject is administered the DNA vaccine (e.g., pTVG-HP) and the PD-1 pathway inhibitor on the same day to initiate treatment (e.g., “Day 1”), e.g., the PD-1 pathway inhibitor is administered from 0 to 0.5 to 5 hours and up to 24 hours after administration of the DNA vaccine. Then, in some embodiments, the DNA vaccine and/or the PD-1 inhibitor is/are administered on several subsequent days after initiation of the treatment. For example, in some embodiments the DNA vaccine is administered on Day 15 (e.g., 15±3 days (e.g., 12 to 18 days) after the Day 1 administration of the DNA vaccine and the PD-1 inhibitor), on Day 29 (e.g., 14±3 days (e.g., 11 to 17 days) after the Day 15 administration of the DNA vaccine), on Day 43 (e.g., 14±3 days (e.g., 11 to 17 days) after the Day 29 administration of the DNA vaccine), on Day 57 (e.g., 14±3 days (e.g., 11 to 17 days) after the Day 43 administration of the DNA vaccine), and on Day 71 (e.g., 14±3 days (e.g., 11 to 17 days) after the Day 57 administration of the DNA vaccine); and the PD-1 inhibitor is administered on Day 22 (e.g., 7±3 days (e.g., 4 to 10 days) after the Day 15 administration of the DNA vaccine), on Day 43 (e.g., 0 to 0.5 to 5 hours after the Day 43 administration of the DNA vaccine), and on Day 64 (e.g., 7±3 days (e.g., 4 to 10 days) after the Day 57 administration of the DNA vaccine).

In some preferred embodiments, the vaccine and the PD-1 inhibitor are administered a plurality of times in an overlapping administration schedule. In some embodiments, the first time the vaccine and the PD-1 inhibitor are administered concurrently (i.e., at day 1 of the treatment schedule within 24 hours of one another) and thereafter the vaccine is administered every 10 to 20 or 21, preferably about every 14 days and the PD-1 inhibitor is administered every 17 to 24 days, preferably about every 21 days for a period of up to 90 days. In some embodiments, the methods further comprise administering the vaccine every 10 to 20 or 21 days, preferably about every 14 days, and the PD-1 inhibitor every 17 to 24 days, preferably about every 21 days, for a period of from 91 days to 365 days. In some embodiments, patients that exhibit a decrease in PSA or tumor regression after 90 days are selected for the administration of the vaccine every 10 to 20 or 21 days and the PD-1 inhibitor every 17 to 24 days for a period of from 91 days to 365 days. In some embodiments, the methods further comprise administering the vaccine every 10 to 20 or 21 days, preferably about every 14 days, and the PD-1 inhibitor every 17 to 24 days, preferably about every 21 days, for a period of from 366 days to 730 days. In some embodiments, patients that exhibit a decrease in PSA or tumor regression after 365 days are selected for the administration of the vaccine every 10 to 20 or 21 days and the PD-1 inhibitor every 17 to 24 days for a period of from 366 days to 730 days. In some embodiments, the vaccine and the PD-1 inhibitor are administered in an overlapping schedule every 10 to 28 days, preferably every 10 to 20 or 21 or 21 days or 10 to 24 days, and most preferably about every 14 days or 21 days for a period of up to 90 days. In some embodiments, the methods further comprise administering the vaccine and the PD-1 inhibitor in an overlapping schedule every 10 to 28 days, preferably every 10 to 20 or 21 days or 10 to 24 days, and most preferably about every 14 days for a period of from 91 days to 365 days. In some embodiments, patients that exhibit a decrease in PSA or tumor regression after 90 days are selected for the administration of the vaccine and the PD-1 inhibitor in an overlapping schedule every 10 to 28 days, preferably every 10 to 20 or 21 days or 10 to 24 days, and most preferably about every 14 days for a period of from 91 days to 365 days. In some embodiments, the methods further comprise administering the vaccine and the PD-1 inhibitor in an overlapping schedule every 10 to 28 days, preferably every 10 to 20 or 21 days or 10 to 24 days, and most preferably about every 14 days for a period of from 366 days to 730 days. In some embodiments, patients that exhibit a decrease in PSA or tumor regression after 365 days are selected for the concurrent administration of the vaccine and the PD-1 inhibitor every 10 to 28 days, preferably every 10 to 20 or 21 days or 10 to 24 days, and most preferably about every 14 days for a period of from 366 days to 730 days. In some embodiments, the vaccine and PD-1 inhibitor are administered concurrently within the overlapping administration schedule. Concurrent administration encompasses administering the vaccine and the PD-1 inhibitor in the same composition (e.g., a solution), or where the agents are administered separately, administration in the same day and preferably administration within from about 1 minute to about 5 hours or 24 hours of one another or from about 30 minutes to about 5 hours or 24 hours of one another.

In other embodiments of the dosing schedule, a subject is administered the DNA vaccine (e.g., pTVG-HP) to initiate treatment (e.g., “Day 1”) and is not administered the PD-1 inhibitor on Day 1. Then, the DNA vaccine and/or the PD-1 inhibitor is/are administered on several subsequent days after initiation of the treatment. For example, in some embodiments the DNA vaccine is administered on Day 15 (e.g., 15±3 days (e.g., 12 to 18 days) after the Day 1 administration of the DNA vaccine), on Day 29 (e.g., 14±3 days (e.g., 11 to 17 days) after the Day 15 administration of the DNA vaccine), on Day 43 (e.g., 14±3 days (e.g., 11 to 17 days) after the Day 29 administration of the DNA vaccine), on Day 57 (e.g., 14±3 days (e.g., 11 to 17 days) after the Day 43 administration of the DNA vaccine), and on Day 71 (e.g., 14±3 days (e.g., 11 to 17 days) after the Day 57 administration of the DNA vaccine); and the PD-1 inhibitor is administered after the series of administrations of the DNA vaccine, e.g., on Day 85 (e.g., 14±3 days (e.g., 11 to 17 days) after the Day 71 administration of the DNA vaccine), on Day 106 (e.g., 21±3 days (e.g., 18 to 24 days) after the Day 85 administration of the PD-1 inhibitor), on Day 127 (e.g., 21±3 days (e.g., 18 to 24 days) after the Day 106 administration of the PD-1 inhibitor), and on Day 148 (e.g., 21±3 days (e.g., 18 to 24 days) after the Day 127 administration of the PD-1 inhibitor). See, e.g., FIG. 1.

In some embodiments, during any time of the dosing schedule, one or more tests may be performed on the subject or using a sample obtained from the subject. Exemplary tests include one or more tests, e.g., to measure the levels of chemicals, biomarkers, metabolites, etc. (e.g., sodium, potassium, bicarbonate, BUN, creatinine, glucose, ALT, AST, bilirubin, alkaline phosphatase, amylase, thyroid stimulating hormone (TSH), LDH, serum prostate specific antigen (PSA), serum PAP, serum testosterone; blood tests (e.g., CBC, e.g., including, in some embodiments, a differential and platelet count); and other tests including, e.g., CT scan; bone scan; physical examination; leukapheresis; antibody panel; CTC counts; tissue biopsy; pulse; blood pressure; respiratory rate; body temperature; T-cell response; PET scan; questionnaire; etc.

In some embodiments, the AR antagonist is added to the above dosing schedules at an intermittent frequency. For example, in some embodiments, the AR antagonist is administered for a period of several weeks or months (e.g., one or more times daily for 1, 2, 3, 4, 5, 6, 8, 10, 12, 14, 18, 20 weeks or more) following by a break in dosing (e.g., a break of 1, 2, 3, 4, 5, 6, 8, 10, 12, 14, 18, 20 weeks or more) in which the DNA vaccine an PD-1 inhibitor are administered at a schedule described herein. For example, in some embodiments, the AR antagonist is administered for a period of 90 days every 90 days (e.g., 90 days of daily dosing with a 90 day break) for the duration of treatment (e.g., 1 month, 6 month, 1 year or more), although other schedules are specifically contemplated.

### Response to Therapy and Monitoring

In some embodiments, the response to therapy with the combination therapy described herein causes a decrease in size of a neoplastic lesion, decreases the biological tumor burden of the subject, etc. For example, in some embodiments a subject's “measurable” lesions are identified and monitored prior to, during, and after treatment. In some embodiments, the subject's tumor burden after initial treatment is evaluated to set a baseline for monitoring the course of treatment, e.g., to provide a measurement to which subsequent measurements are compared. In some embodiments, “baseline” tumor burden is determined by imaging the subject. As used herein, a subject has “measurable disease” when the subject has at least one “measurable lesion”. As used herein, a “measurable lesion” is a lesion that can be accurately measured in at least one dimension (longest diameter to be recorded) as >20 mm (2.0 cm) with conventional techniques or as >10 mm (1.0 cm) with spiral CT scan. For lymph node metastases, a “measurable lesion” is at least 2.0 cm in longest diameter by spiral CT or conventional techniques. In some embodiments, tumor lesions that are situated in a previously irradiated area are not considered measurable. As used herein, a “non-measurable” lesion is a lesion that does not “measureable”, e.g., all other lesions, including small lesions (longest diameter <20 mm (2.0 cm) with conventional techniques or <10 mm (1.0 cm) with spiral CT scan), lymph nodes <2.0 cm, and lesions for which measurements cannot be obtained reliably (e.g., bone lesions, leptomeningeal disease, ascites, pleural/pericardial effusion, lymphangitis cutis/pulmonis, abdominal masses that are not confirmed and followed by imaging techniques, and cystic lesions).

In some embodiments, a lesion is measured with positron emission tomography (PET) (e.g., ¹⁸F NaF PET). ¹⁸F NaF PET finds use in producing 3-dimensional measurements of, e.g., tissues, and thus provides a volumetric quantification of lesion volumes and therefore a measurement of total tumor volume.

In some embodiments, response to therapy is monitored by monitoring the sizes of measurable lesions representative of involved organs. In some embodiments, RECIST 1.1 is used for evaluation of radiographic data. In some embodiments, Immune related Response Criteria (irRC) based on WHO criteria are used for evaluation of immune-oncology, e.g., the irRECIST criteria that are based on RECIST 1.1, irRC, and Nishino (2013) “Developing a common language for tumor response to immunotherapy: immune-related response criteria using unidimensional measurements.”, Clin Cancer Res. 19(14): 3936-43, incorporated herein by reference.

In some embodiments, target lesions are selected on the basis of size (e.g., those with the longest diameters) and their suitability for accurate repeated measurements. In some embodiments, response to therapy is monitored by calculating the sum of the longest diameters of all target lesions to provide a sum longest diameter. In some embodiments, the sum longest diameter is used to characterize the tumor response. For lesions measurable in 2 or 3 dimensions, the longest diameter at the time of each assessment is used. Further, in some embodiments lymph node measurements are made using short axis measurements, e.g., as per RECIST 1.1 and/or irRECIST.

In some embodiments, a subject is placed in a class based on the subject's response to treatment. For instance, a subject is placed in a “Complete Response (CR)” class based on the disappearance of all target lesions. In some embodiments, to be assigned a status of complete response, changes in tumor measurements are confirmed by repeat assessments performed no less than four weeks after the criteria for response are first met. In some embodiments, PSA is <0.2 ng/mL. In some embodiments, lymph nodes that shrink to less than 1.0 cm are considered normal. A subject is placed in a “Partial Response (PR)” class based on at least a 30% decrease in the sum of the longest diameters of target lesions, taking as reference the baseline sum longest diameter. In some embodiments, to be assigned a status of partial response, changes in tumor measurements must be confirmed by repeat assessments performed no less than four weeks after the criteria for response are first met. In some embodiments, there are no new lesions. A subject is placed in a “Progressive Disease (PD)” class based on at least a 20% increase in the sum of the longest diameters of target lesions, and a 0.5 cm absolute minimum increase, taking as reference the smallest sum longest diameter recorded since the baseline measurements, or the appearance of one or more new lesion(s). A subject is placed in a “Stable Disease (SD)” class based on neither sufficient shrinkage to qualify for partial response nor sufficient increase to qualify for progressive disease. In some embodiments, to be assigned a status of stable disease, measurements meet the stable disease criteria at least once after study entry at a minimum interval of 12 weeks.

In some embodiments, monitoring the response to treatment comprises monitoring non-target lesions, e.g., all lesions or sites of disease that are not target lesions. In some embodiments, non-target lesions are monitored, e.g., by bone scintigraphy. In some embodiments, a subject is placed into a subject class based on monitoring of non-target lesions. For instance, a subject is placed in a “Complete Response (CR)” class based on the disappearance of all non-target lesions and undetectable PSA tumor marker levels. In some embodiments, to be assigned a status of complete response, changes in tumor measurements are confirmed by repeat assessments performed no less than four weeks after the criteria for response are first met. In some embodiments, a subject is placed in a “Incomplete Response/Stable Disease (SD)” class based on the persistence of one or more non-target lesion(s) and/or the persistence of detectable serum PSA tumor marker levels. In some embodiments, to be assigned a status of stable disease, measurements meet the stable disease criteria at least once after study entry at a minimum interval of 12 weeks. In some embodiments, a subject is placed in a “Progressive Disease (PD)” class based on the appearance of one or more new lesion(s) and/or unequivocal progression of existing nontarget lesions. In some embodiments, for lesions only detectable by bone scan, the appearance of >2 new lesions, with symptoms, constitutes disease progression. Without symptoms, and if no other evidence of disease progression (e.g., no progressive disease by PSA or measurable disease criteria), in some embodiments progression is documented with bone scintigraphy, e.g., at least 6 weeks later demonstrating >2 new lesions, to eliminate the possibility of flair responses seen on bone scans.

In some embodiments, PSA progression (e.g., based on PSA amounts and/or kinetics) is monitored to monitor a subject's response to treatment. In some embodiments, a subject is classified as “PSA Complete Response” based on a decrease in PSA to <0.2 ng/mL. In some embodiments, placement in the “PSA Complete Response” class is confirmed with a later PSA measurement, e.g., at a minimum of four weeks later (confirmed PSA CR). In some embodiments, placement in the “PSA Complete Response” is further based on no evidence of radiographic progression. In some embodiments, a subject is placed in a “PSA Partial Response” class based on a greater than or equal to 50% reduction in baseline PSA. In some embodiments, placement in the “PSA Partial Response” class comprises no evidence of radiographic progression. In some embodiments, time to PSA progression is used to monitor a subject's response to treatment. As used herein, PSA progression refers to a 50% increase in PSA over the nadir PSA, and >2 ng/mL above the nadir, confirmed by a second value 3 or more weeks later (e.g., a confirmed rising trend). If no on-study reduction has occurred, nadir is baseline value (e.g., pre-treatment).

In some embodiments, subjects' immunological systems are monitored, e.g., by assessing PAP- or AR-specific CD8+ T-cell effector immunity, by assessing PAP- or AR-specific memory T-cell immunity, by assessing PAP- or AR-specific T cells, by assessing antigen-specific antibodies (e.g., PAP- or AR-specific antibodies), by assessing antigen-specific regulatory immune responses, and/or by assessing antigen-spread to other prostate-associated antigens. In some embodiments, a subject is evaluated by enumerating and characterizing circulating tumor cells. In some embodiments, a subject is evaluated using histology, e.g., by examining a tissue biopsy. In some embodiments, a subject is evaluated by quantitative total bone imaging using PET and/or CT (e.g., NaF PET/CT).

In some embodiments, subjects are evaluated using total bone imaging (e.g., quantitative total bone imaging, QTBI) using NaF PET/CT. For example, in some embodiments an assessment of small volume bone metastatic disease and tumor growth rates are conducted using QTBI. For example, in some embodiments, the subject has a bone disease that is not detected by standard bone scintigraphy. In some embodiments, patients are assessed at various time points, e.g., at 1 month prior to treatment, at baseline, and at 3 months on treatment. In some embodiments, metastatic prostate cancer lesions in bone are localized and identified based on functional NaF PET uptake, assisted with the anatomical information provided by CT scans. In some embodiments, segmentation is performed using an automatic segmentation method (e.g., using a fixed SUV threshold), and adjusted with physician guidance. In some embodiments, scans from different time points are registered to one another using an articulated registration technique employing a rigid registration of skeletal elements (e.g., bones) from CT followed by registration optimization by combining with deformable registration of bones and lesions from NaF PET/CT. In some embodiments, the lesions between pre-treatment and follow-up scans are matched to establish longitudinal correspondence of lesions. For each patient, in some embodiments, comprehensive treatment response metrics are calculated, comprising, e.g., SUVₜₒₜₐₗ (total disease burden), SUVₘₐₓ (maximum intensity lesion), SUVₘₑₐₙ (average intensity), the number of lesions, and total volume of bone lesions. In addition, imaging response metrics are calculated in some embodiments for each individual lesion. In some embodiments, this methodology is used to assess the growth rate of bone metastatic disease by evaluating changes over time, e.g., from pre-treatment to baseline and comparing this to measurements made from baseline to month 3.

### Kits

In some embodiments, the technology provides kits for treating a subject having prostate cancer or a subject at risk for prostate cancer. For example, some embodiments provide a first composition (e.g., a first pharmaceutical composition) comprising a nucleic acid (e.g., DNA) vaccine (e.g., comprising a nucleic acid comprising a nucleotide sequence from a PAP and/or AR gene) and a second composition (e.g., a second pharmaceutical composition) comprising a PD-1 inhibitor and a third composition comprising an AR antagonist. In addition, some kit embodiments further comprise a product insert providing a dosing schedule comprising instructions relating to the administration of the nucleic acid vaccine, the PD-1 inhibitor, and the AR antagonist.

In some embodiments, the nucleic acid vaccine comprises pTVG-HP and the PD-1 inhibitor is a monoclonal antibody inhibitor of PD-1. In some embodiments, the nucleic acid vaccine comprises pTVG-HP and the PD-1 inhibitor is pembrolizumab. In some embodiments, the nucleic acid vaccine comprises pTVG-HP and the PD-1 inhibitor is nivolumab. In some embodiments, the DNA vaccine comprises an adjuvant, e.g., GM-CSF. In some embodiments, the AR antagonist is enzalutamide or apalutamide.

In some embodiments, the DNA vaccine, the PD-1 inhibitor, and the AR antagonist are provided as ready-to-use pharmaceutical compositions. In some embodiments, the DNA vaccine, the PD-1 inhibitor, and the AR antagonist are provided in a dried (e.g., lyophilized) state, e.g., for solubilization and/or resuspension in a pharmaceutically appropriate solution prior to administration.

In some embodiments, the kits comprise the DNA vaccine, the PD-1 inhibitor, and the AR antagonist in a vessel such as a vial, ampule, bottle, etc. In some embodiments, the DNA vaccine, the PD-1 inhibitor, and the AR antagonist are provided in single-dose amounts in a vessel such as a vial, ampule, bottle, etc. For instance, some kit embodiments comprise: 1) a first vial comprising a pharmaceutical composition comprising approximately 100 μg of the DNA vaccine (e.g., pTVG-HP) (e.g., as a single or multiple DNA vaccines targeting PAP and AR) and approximately 208 μg of GM-CSF; 2) a second vial comprising from 10 mg to 1000 mg of PD-1 inhibitor; and 3) a third vial comprising enzalutamide or apalutamide. In some embodiments, the first vial comprises approximately 200 to 300 μl of the pharmaceutical composition. In some embodiments, kits comprise two vials of the DNA vaccine to provide two doses of the DNA vaccine.

Some embodiments of the kits provide multiple doses of the DNA vaccine, the PD-1 inhibitor, and the AR antagonist, e.g., to provide a sufficient number of doses to complete a dosing schedule as described herein. For example, some embodiments of kits comprise 5 to 20 vials of the DNA vaccine and some embodiments of kits comprise 2 to 10 vials of the PD-1 inhibitor and the AR antagonist.

### EXAMPLES

### Example 1—Clinical Trial Protocol

This Example describes a clinical trial protocol for testing combination therapies in prostate cancer. The trial tests the hypothesis that immunization targeting one or two prostate cancer antigens, in combination PD-1 blockade and apalutamide, leads to effective anti-tumor immunity as demonstrated by PSA complete responses that persist after discontinuation of the apalutamide. The ability to drive PSA to undetectable levels, possibly curing or significantly delaying the metastatic recurrence of prostate cancer prior to the need for androgen depriving therapies, is a substantial and clinically meaningful “game-changing” advance in the therapy of this disease.

The trial is conducted as a randomized Phase 2 multi-institution study in this population. Specifically, the trial evaluates the use of one versus two DNA vaccines targeting different antigens (PAP and AR), delivered concurrently with PD-1 blockade using JNJ-63723283, in a regimen that includes a 12-week course of apalutamide, over a total 6-month period.

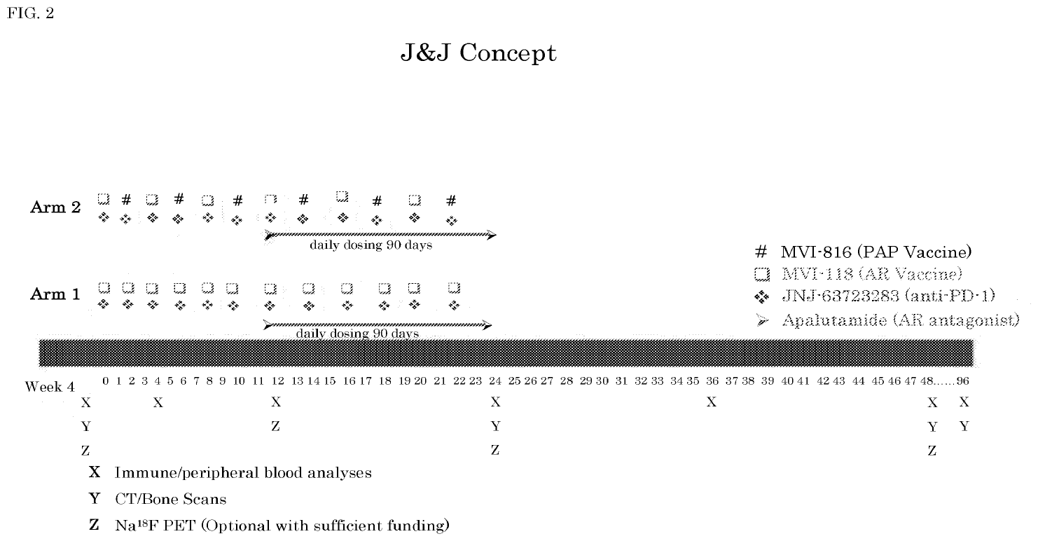

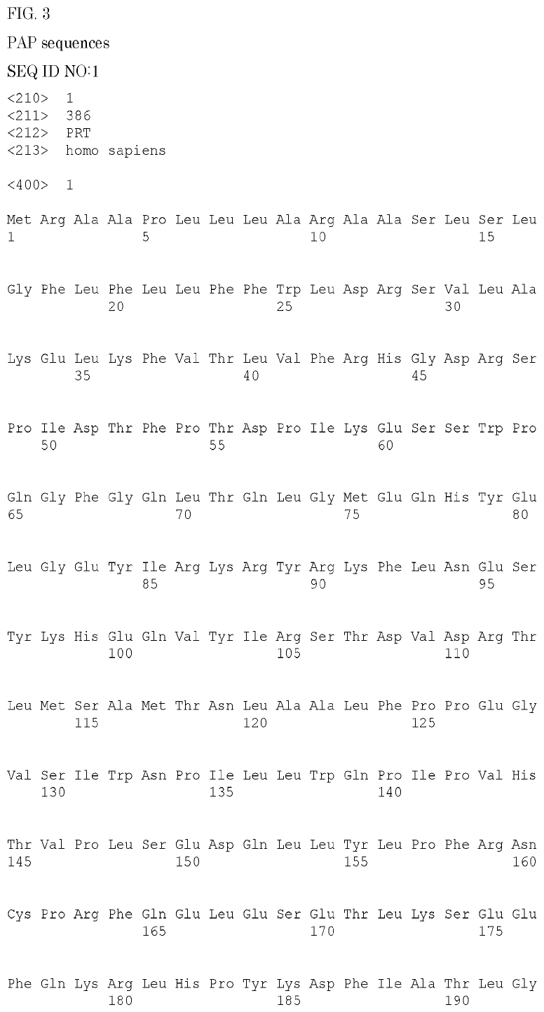

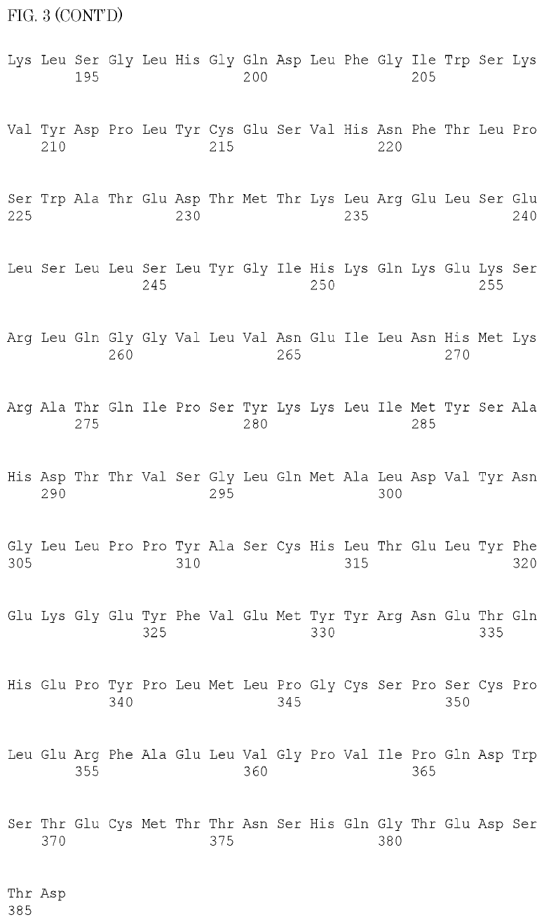

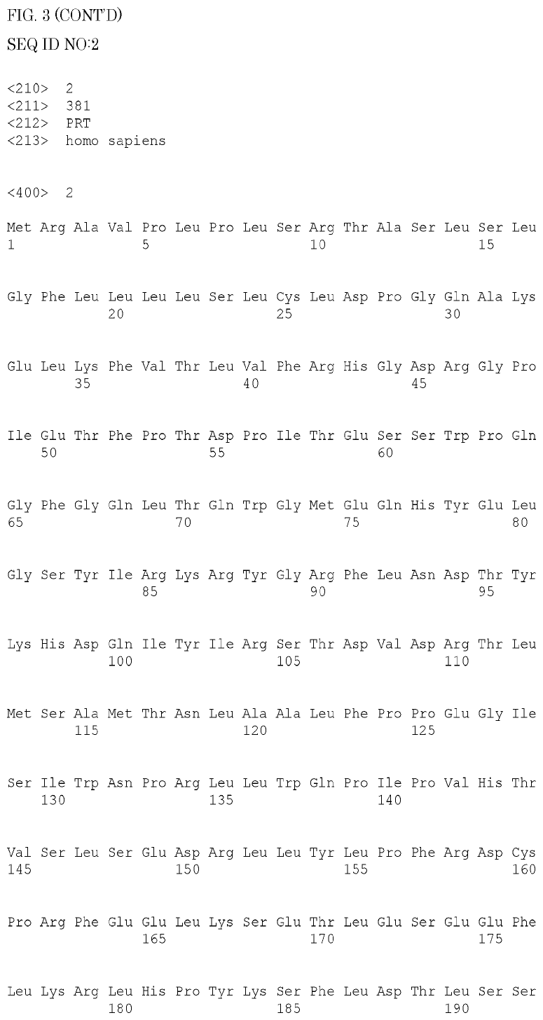

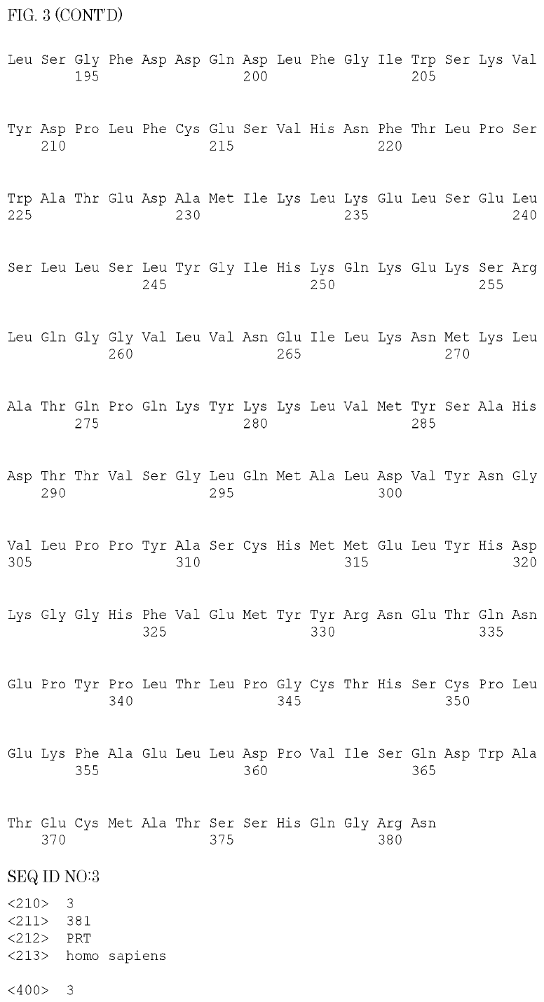

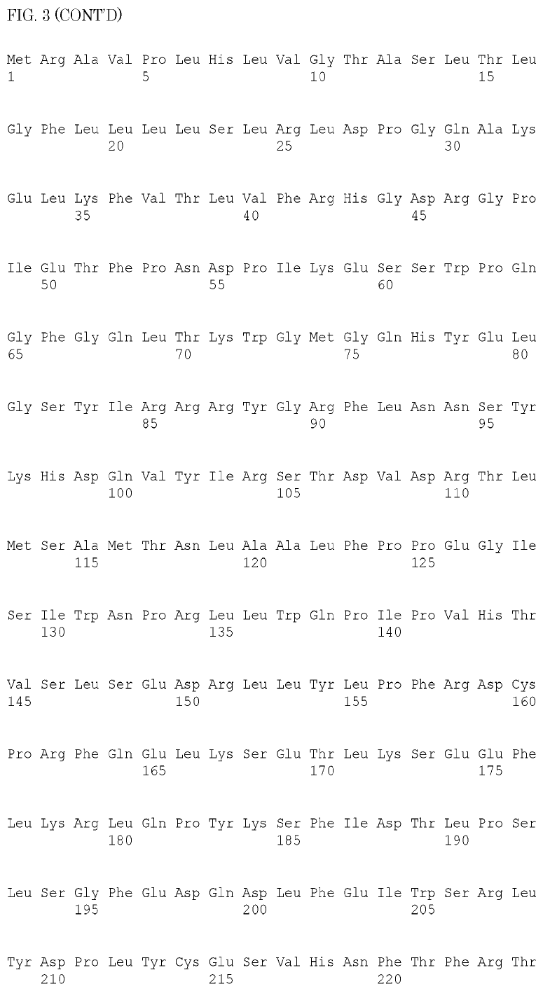

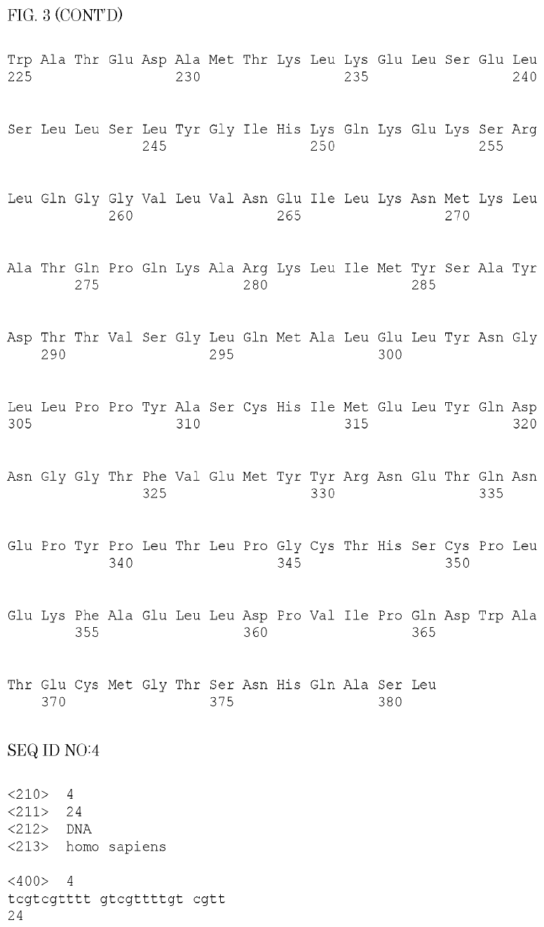

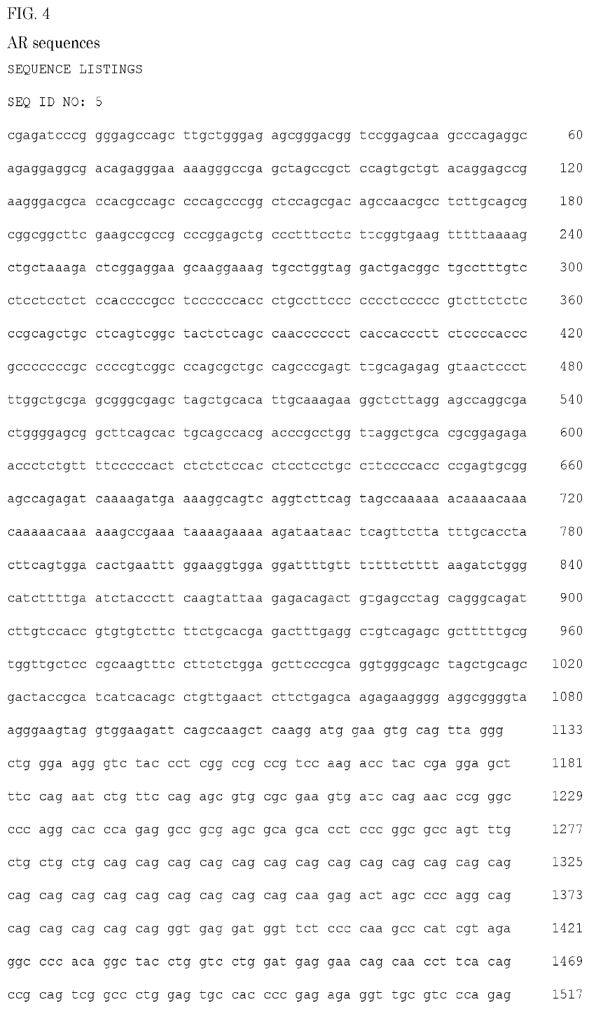

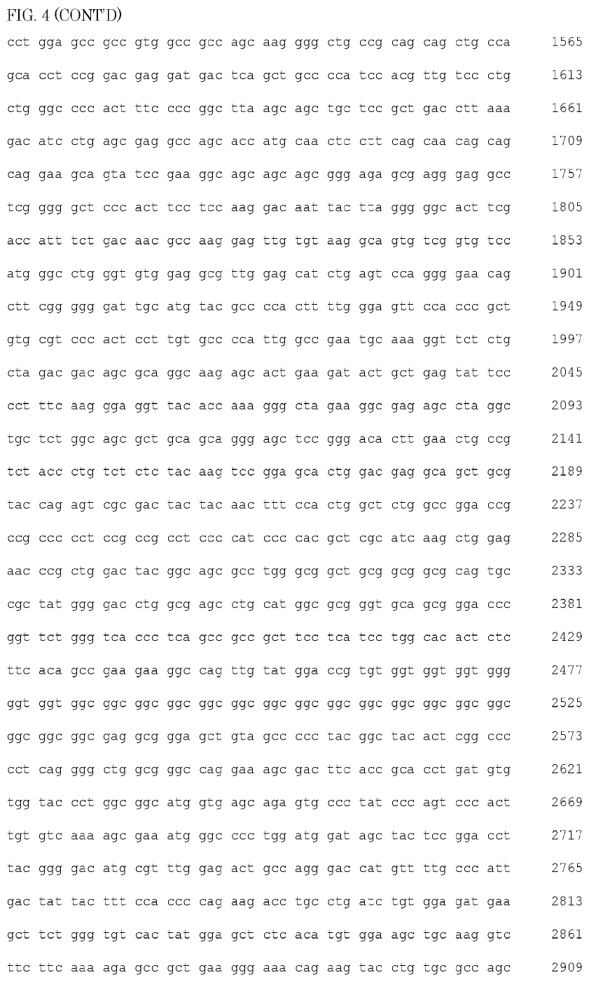

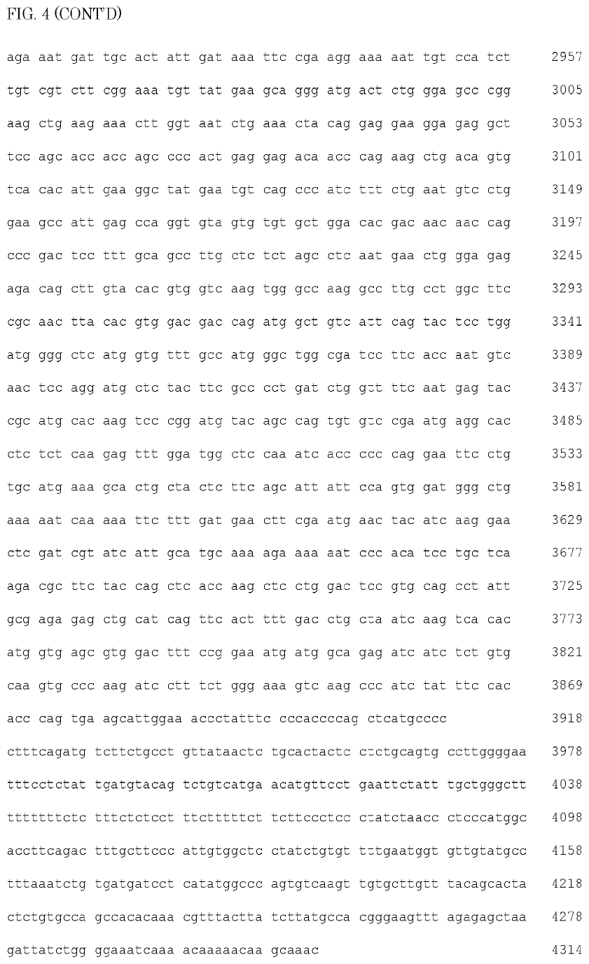

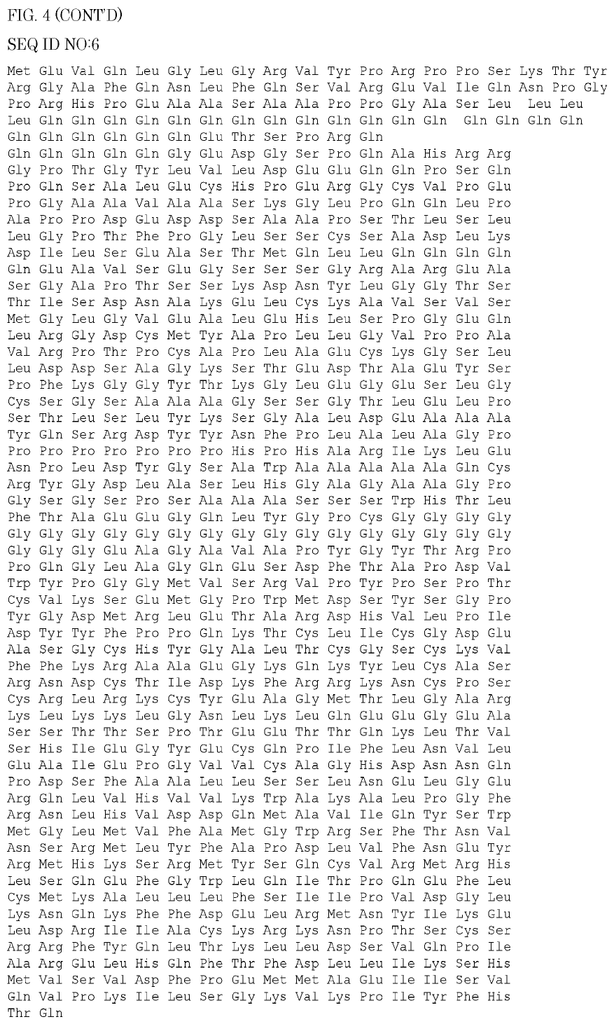

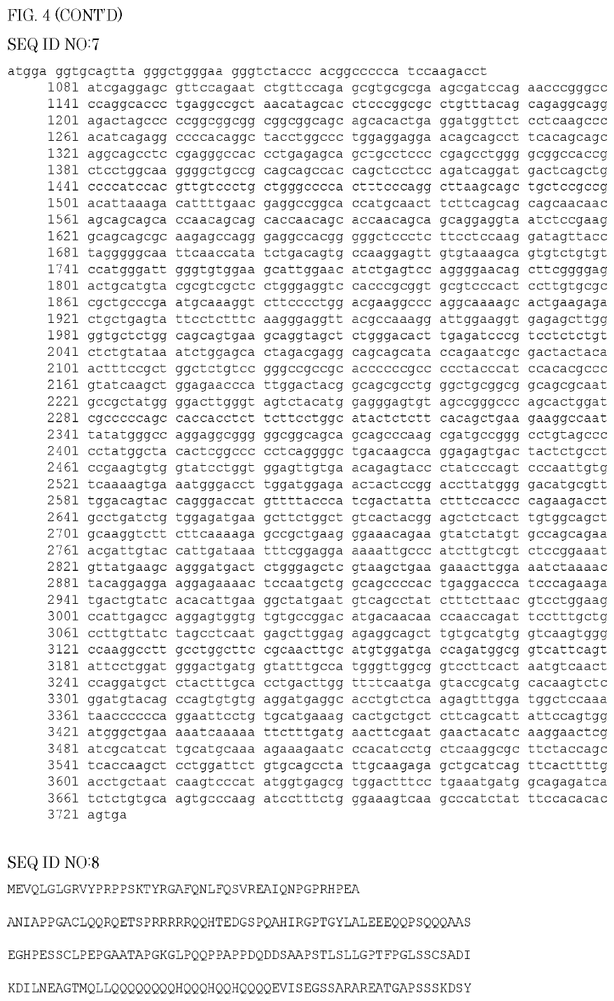

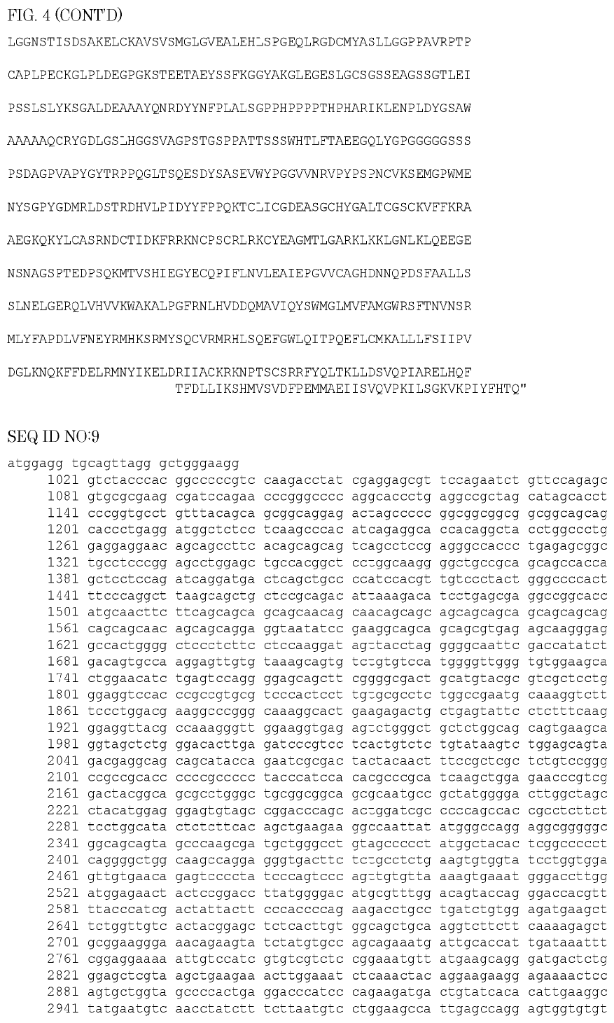

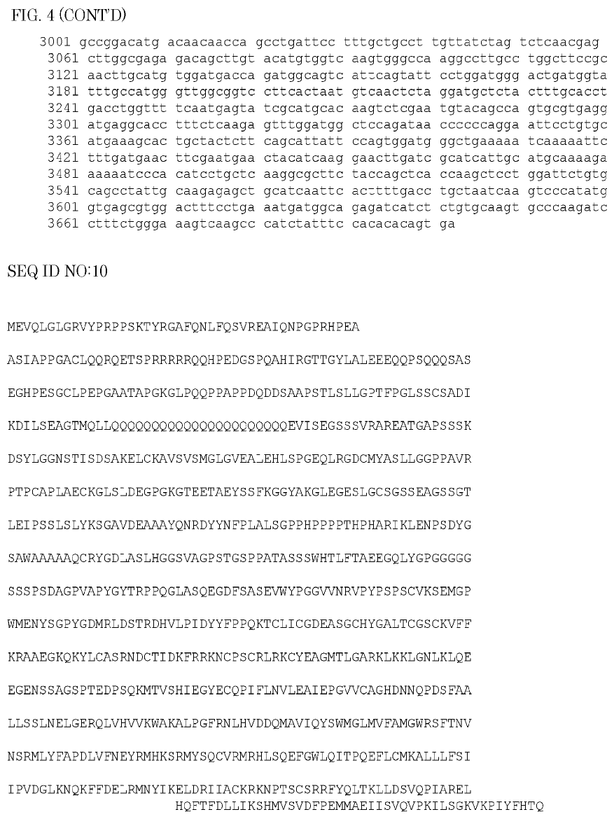

The study protocol is shown in FIG. 2. The trial evaluates the safety and tolerability of MVI-118 DNA vaccine+/−MVI-816 DNA vaccine and JNJ-63723283 (Janssen, Raritan, N.J.) and apalutamide in patients with DO/M0 prostate cancer. One-year PSA complete response (PSA<0.2 ng/ml) rates are measured.

The trial further evaluates 2-year metastasis-free survival rate and median radiographic progression-free survival. Additional experiments determine if antigen-specific T-cell and/or IgG responses are elicited with treatment and if NaF PET/CT can be used to monitor tumor response.

### Example 2—Immune Response to AR

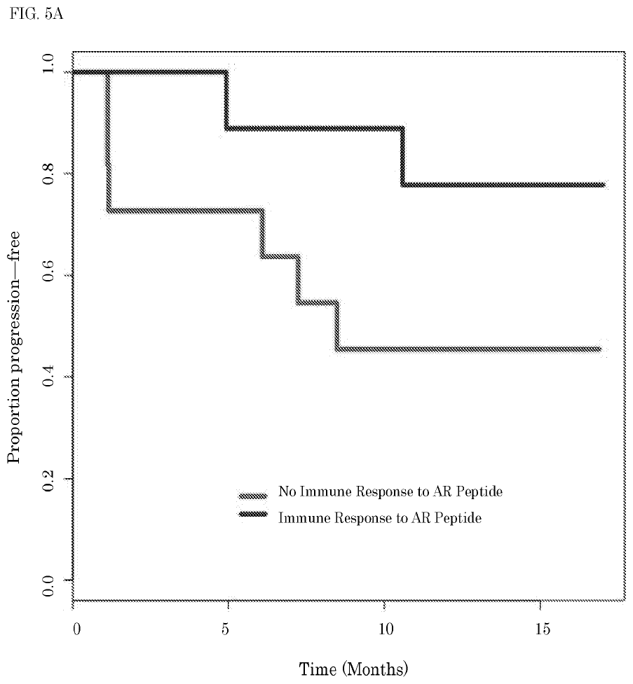

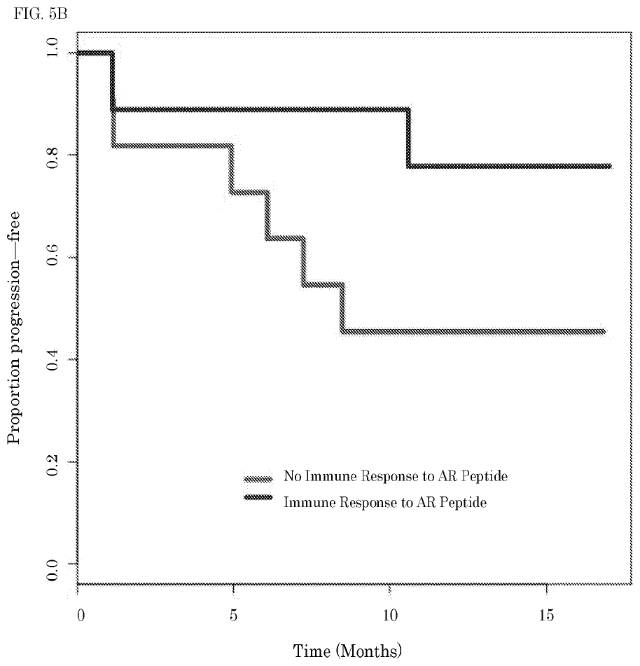

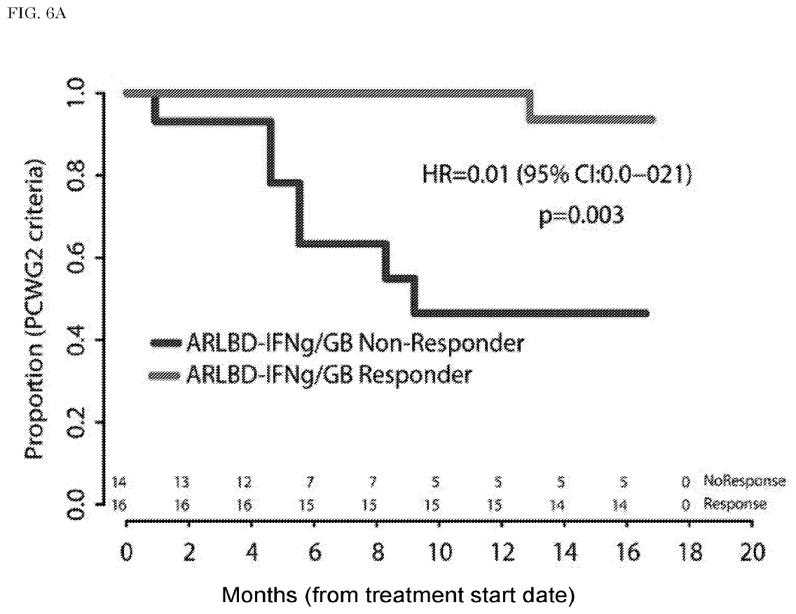

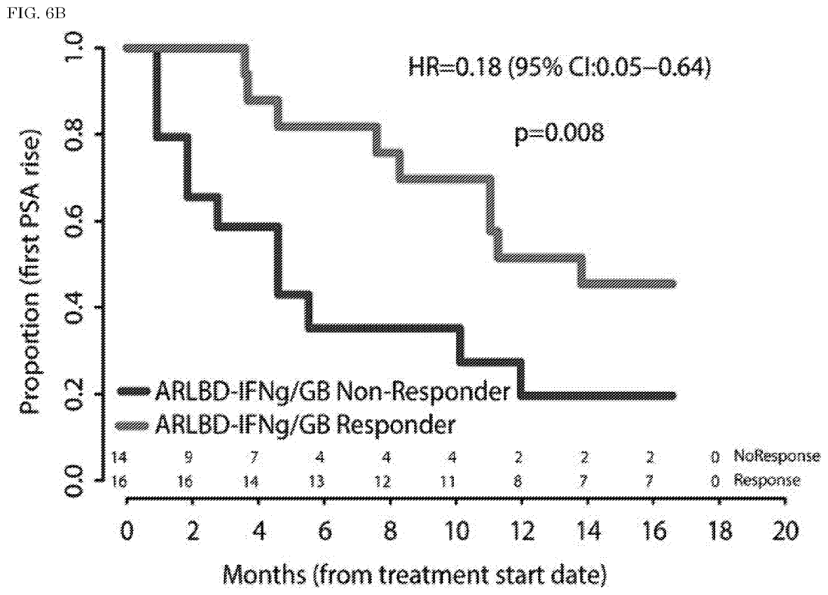

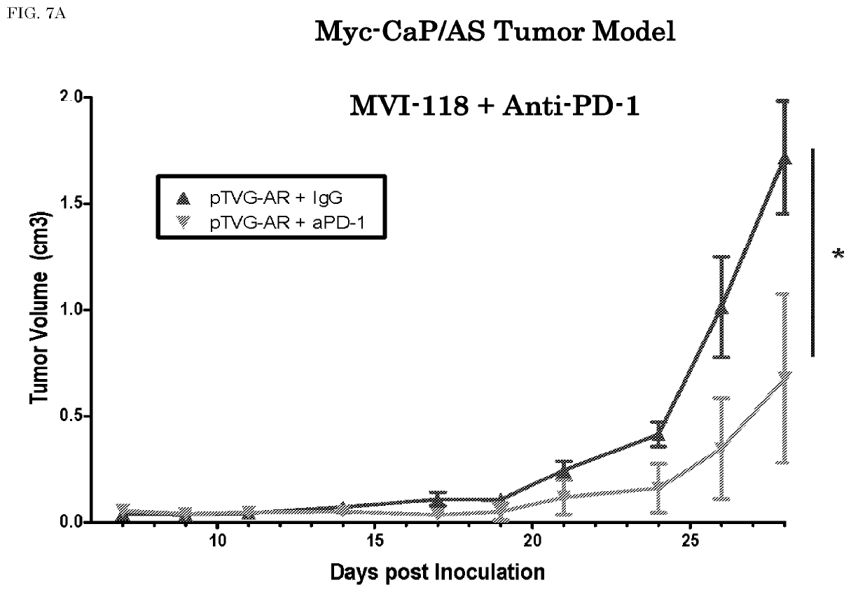

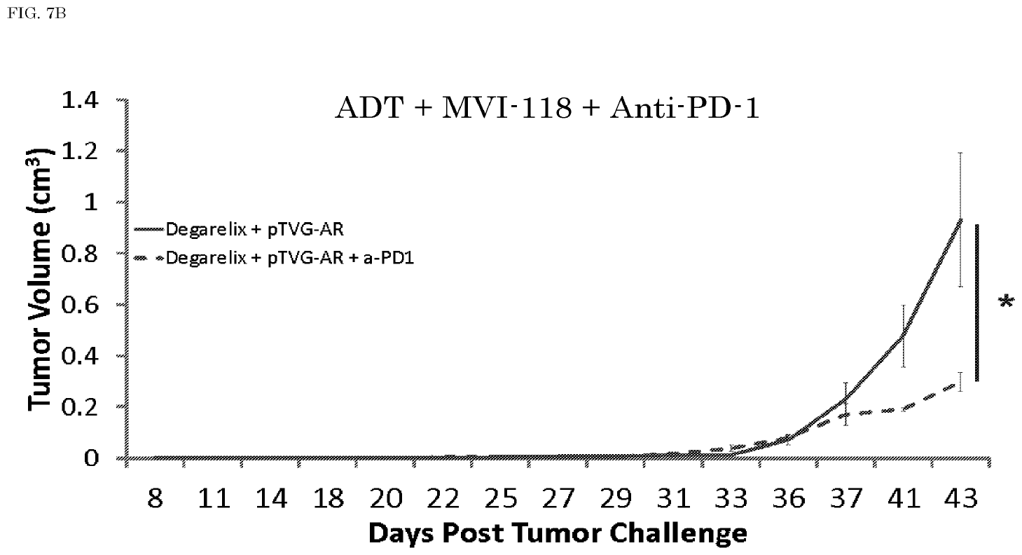

FIG. 5 shows progression free survival in subjects with and without immune response to A) AR peptide or B) protein. FIG. 6 shows data for the AR vaccine in combination with ADT showing a positive correlation between patient immune response to AR and delayed time to PSA progression. FIG. 7 shows that a PD-1 inhibitor in combination with AR vaccine or AR vaccine plus ADT shows improved efficacy in mouse tumor models.

Table 1 shows PSA Progression: Date from “on study date” to date of PSA progression (defined as PSA increase >25% over pre-treatment or an absolute increase by 2 ng/mL) or “off-study date (if there was no PSA progression).

TABLE 1
Summary of PSA Progression-free survival

| | | | | Median | Hazard | | |
|---|---|---|---|---|---|---|---|
| | | N | Events | (months) | Ratio | 95% CI | p-value |
| Immune Response | Yes | 9 | 2 | NBR^{†} | 0.32 | 0.07-1.61 | 0.1445 |
| to AR Protein | No | 11 | 6 | 8.5 | | | |
| Immune Response | Yes | 9 | 2 | NBR^{†} | 0.31 | 0.06-1.52 | 0.1236 |
| to AR Peptide | No | 11 | 6 | 8.5 | | | |
| Library | | | | | | | |

^{†}Not been reached after a median follow-up of 14 months

### References:

1. Couzin-Frankel, J. (2013). “Breakthrough of the year 2013. Cancer immunotherapy.” Science. 342:1432-3.
2. Topalian, S. L., F. S. Hodi, J. R. Brahmer, S. N. Gettinger, D. C. Smith, D. F. McDermott, J. D. Powderly, R. D. Carvajal, J. A. Sosman, M. B. Atkins, P. D. Leming, D. R. Spigel, S. J. Antonia, L. Horn, C. G. Drake, D. M. Pardoll, L. Chen, W. H. Sharfman, R. A. Anders, J. M. Taube, T. L. McMiller, H. Xu, A. J. Korman, M. Jure-Kunkel, S. Agrawal, D. McDonald, G. D. Kollia, A. Gupta, J. M. Wigginton, and M. Sznol. (2012). “Safety, activity, and immune correlates of anti-PD-1 antibody in cancer.” N Engl J Med. 366:2443-54.
3. Sfanos, K. S., T. C. Bruno, A. K. Meeker, A. M. De Marzo, W. B. Isaacs, and C. G. Drake. (2009). “Human prostate-infiltrating CD8+T lymphocytes are oligoclonal and PD-1+.” Prostate. 69:1694-703.
4. McNeel, D. G., E. J. Dunphy, J. G. Davies, T. P. Frye, L. E. Johnson, M. J. Staab, D. L. Horvath, J. Straus, D. Alberti, R. Marnocha, G. Liu, J. C. Eickhoff, and G. Wilding. (2009). “Safety and immunological efficacy of a DNA vaccine encoding prostatic acid phosphatase in patients with stage DO prostate cancer.” J Clin Oncol. 27:4047-54.
5. McNeel, D. G., J. T. Becker, J. C. Eickhoff, L. E. Johnson, E. Bradley, I. Pohlkamp, M. J. Staab, G. Liu, G. Wilding, and B. M. Olson. (2014). “Real-time immune monitoring to guide plasmid DNA vaccination schedule targeting prostatic acid phosphatase in patients with castration-resistant prostate cancer.” Clin Cancer Res. 20:3692-704.
6. Becker, J. T., B. M. Olson, L. E. Johnson, J. G. Davies, E. J. Dunphy, and D. G. McNeel. (2010). “DNA vaccine encoding prostatic acid phosphatase (PAP) elicits long-term T-cell responses in patients with recurrent prostate cancer.” J Immunother. 33:639-47.
7. Rekoske, B. T., B. M. Olson, and D. G. McNeel. (2016). “Antitumor vaccination of prostate cancer patients elicits PD-1/PD-L1 regulated antigen-specific immune responses.” Oncoimmunology. 5:e1165377.
8. Topalian, S. L., F. S. Hodi, J. R. Brahmer, S. N. Gettinger, D. C. Smith, D. F. McDermott, J. D. Powderly, R. D. Carvajal, J. A. Sosman, M. B. Atkins, P. D. Leming, D. R. Spigel, S. J. Antonia, L. Horn, C. G. Drake, D. M. Pardoll, L. Chen, W. H. Sharfman, R. A. Anders, J. M. Taube, T. L. McMiller, H. Xu, A. J. Korman, M. Jure-Kunkel, S. Agrawal, D. McDonald, G. D. Kollia, A. Gupta, J. M. Wigginton, and M. Sznol. (2012). “Safety, activity, and immune correlates of anti-PD-1 antibody in cancer.” N Engl J Med. 366:2443-54.
9. Brahmer, J. R., C. G. Drake, I. Wollner, J. D. Powderly, J. Picus, W. H. Sharfman, E. Stankevich, A. Pons, T. M. Salay, T. L. McMiller, M. M. Gilson, C. Wang, M. Selby, J. M. Taube, R. Anders, L. Chen, A. J. Korman, D. M. Pardoll, I. Lowy, and S. L. Topalian. (2010). “Phase I study of single-agent anti-programmed death-1 (MDX-1106) in refractory solid tumors: safety, clinical activity, pharmacodynamics, and immunologic correlates.” J Clin Oncol. 28:3167-75.
10. Beer T M, Kwon E D, Drake C G, et al. Randomized, Double-Blind, Phase III Trial of Ipilimumab Versus Placebo in Asymptomatic or Minimally Symptomatic Patients With Metastatic Chemotherapy-Naive Castration-Resistant Prostate Cancer. J Clin Oncol. January 2017; 35(1):40-47.
11. Mercader M, Bodner B K, Moser M T, Kwon P S, Park E S, Manecke R G, et al. T cell infiltration of the prostate induced by androgen withdrawal in patients with prostate cancer. Proc Natl Acad Sci USA 2001; 98:14565-70.
12. Morse M D, McNeel D G. Prostate cancer patients treated with androgen deprivation therapy develop persistent changes in adaptive immune responses. Human Immunol 2010; 71:496-504.
13. Morse M D, McNeel D G. T-cells localized to the androgen-deprived prostate are TH1 and TH17 biased. Prostate 2012; 72:1239-47.
14. Roden A C, Moser M T, Tri S D, Mercader M, Kuntz S M, Dong H, et al. Augmentation of T cell levels and responses induced by androgen deprivation. J Immunol 2004; 173:6098-108.
15. Mercader M, Sengupta S, Bodner B K, Manecke R G, Cosar E F, Moser M T, et al. Early effects of pharmacological androgen deprivation in human prostate cancer. BJU Int 2007; 99:60-7.
16. Gannon P O, Poisson A O, Delvoye N, Lapointe R, Mes-Masson A M, Saad F. Characterization of the intra-prostatic immune cell infiltration in androgen-deprived prostate cancer patients. J Immunol Methods 2009; 348:9-17.
17. Shen Y C, Kochel C, Francica B, Alme A, Nirschl C, Nirschl T, et al. Combining androgen deprivation with immune checkpoint blockade delays the development of castration resistance in a murine model of prostate cancer. 2015; Philadelphia, Pa.
18. Akins E J, Moore M L, Tang S, Willingham M C, Tooze J A, Dubey P. In situ vaccination combined with androgen ablation and regulatory T-cell depletion reduces castration-resistant tumor burden in prostate-specific pten knockout mice. Cancer Res 2010; 70:3473-82.
19. Drake C G, Doody A D, Mihalyo M A, Huang C T, Kelleher E, Ravi S, et al. Androgen ablation mitigates tolerance to a prostate/prostate cancer-restricted antigen. Cancer Cell 2005; 7:239-49.
20. Koh Y T, Gray A, Higgins S A, Hubby B, Kast W M. Androgen ablation augments prostate cancer vaccine immunogenicity only when applied after immunization. Prostate 2009; 69:571-84.
21. Ardiani A, Farsaci B, Rogers C J, Protter A, Guo Z, King T H, et al. Combination therapy with a second-generation androgen receptor antagonist and a metastasis vaccine improves survival in a spontaneous prostate cancer model. Clin Cancer Res 2013; 19:6205-18.
22. Olson B M Gamat M Seliski J Sawicki T Jeffery J Ellis L Drake C G Weichert J McNeel D G Prostate Cancer Cells Express More Androgen Receptor (AR) Following Androgen Deprivation, Improving Recognition by AR-Specific T-cells. Cancer Immunol Res. 2017 December; 5(12):1074-1085
23. McNeel D G, Smith H A, Eickhoff J C, Lang J M, Staab M J, Wilding G, Liu G. Phase I trial of tremelimumab in combination with short-term androgen deprivation in patients with PSA-recurrent prostate cancer. Cancer Immunol Immunother. 2012 July; 61(7):1137-47

All publications and patents mentioned in the above specification (including, but not limited to those cited in the above section) are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method for treating prostate cancer in a subject, the method comprising:
(a) administering to a subject at least one vaccine comprising a nucleic acid comprising a nucleotide sequence selected from the group consisting of a prostatic acid phosphatase (PAP) gene and a ligand-binding domain of an androgen receptor (AR) gene;
(b) administering to the subject a human programmed death receptor-1 (PD-1) inhibitor; and
(c) administering to the subject an androgen receptor antagonist, wherein the vaccine and the PD-1 inhibitor are adminstered concurrently for from 8 to 16 weeks, followed by a period where the vaccine, the PD-1 inhibitor and the androgen receptor antagonist are administered concurrently for from 8 to 16 weeks.

2. The method of claim 1, wherein the concurrent administration comprises administration of the vaccine followed by the administration of the PD-1 inhibitor and/or the androgen receptor antagonist within 24 hours of administration of the vaccine.

3. The method of claim 1, wherein the nucleic acid further comprises a transcriptional regulatory element and/or an immunostimulatory sequence.

4. The method of claim 1, wherein the nucleotide sequence from a PAP or AR gene is operatively linked to a transcriptional regulatory element.

5. The method of claim 1, wherein the PAP gene is a human PAP gene.

6-8. (canceled)

9. The method of claim 1, wherein the nucleic acid is pTVG4-HP.

10. (canceled)

11. The method of claim 1, wherein the PD-1 inhibitor is pembrolizumab, JNJ-63723283, or nivolumab.

12. The method of claim 1, wherein the nucleotide sequence encodes a polypeptide comprising an amino acid sequence from SEQ ID NO: 1, SEQ ID NO: 2, SEQ ID NO: 3, SEQ ID NO:6, or a portion or substituted variant thereof.

13. The method of claim 1, wherein the PD-1 inhibitor is administered at a dose of 1 to 5 mg/kg and the vaccine is administered in an amount of approximately 100 μg.

14. (canceled)

15. The method of claim 1, wherein the androgen receptor antagonist is enzalutamide or apalutamide.

16. The method of claim 15, wherein enzalutamide is administered at a dose of 160 mg and said apalutamide is administered at a dose of 240 mg.

17. The method of claim 1, wherein the vaccine further comprises an adjuvant.

18-20. (canceled)

21. The method of claim 1, wherein the vaccine, the androgen receptor antagonist, and the PD-1 inhibitor are administered a plurality of times, and wherein after the first concurrent administration of the vaccine and the PD-1 inhibitor, the vaccine is administered every 10 to 21 days, the PD-1 inhibitor is administered every 17 to 28 days for a period of up to 90 days, and the androgen receptor antagonist is administered daily for a period of up to 90 days beginning about 8 to 12 weeks after the first administration of the vaccine.

22. The method of claim 21, wherein said androgen receptor antagonist is administered daily for 90 days followed by a period of not administering said androgen receptor antagonist.

23. The method of claim 21, wherein administration of said androgen receptor antagonist is repeated every 90 days.

24. The method of claim 1, wherein the vaccine and the PD-1 inhibitor are administered concurrently every 10 to 28 days for a period of up to 90 days.

25. The method of claim 24, further comprising concurrently administering the vaccine and the PD-1 inhibitor every 10 to 28 days for a period of from 91 days to 365 days.

26. The method of claim 24, further comprising administering the vaccine and the PD-1 inhibitor every 10 to 28 days for a period of from 366 days to 730 days.

27. The method of claim 24, wherein said vaccine comprises a first vaccine to PAP and a second vaccine to AR and said first and second vaccine are administered concurrently.

28. The method of claim 1, wherein the method produces an anti-tumor response in the subject that is improved relative to administration of the vaccine alone or the vaccine in combination with the PD-1 inhibitor.

29-74. (canceled)
